(19) United States
(12) Patent Application Publication
(10) Pub. No.: US 2008/0226588 A1
(43) Pub. Date: Sep. 18, 2008

(54) Treating melanoma with bis(thiohydrazide amides)

(76) Inventor: Matthew McLeod, Boston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/894,270

(22) Filed: Aug. 20, 2007

Related U.S. Application Data
(60) Provisional application No. 60/838,977, filed on Aug. 21, 2006.

(51) Int. Cl. A61K 31/165; A61K 31/337; A61K 33/24; A61K 31/655; A61K 38/21; A61K 38/20; A61K 31/4188; A61K 31/662; A61K 31/4412; A61P 35/00

(52) U.S. Cl. 424/85.2; 514/599; 514/449; 424/649; 514/151; 424/85.4; 514/393; 514/119; 514/350

(57) ABSTRACT

Disclosed herein are methods of treating lentigo maligna, superficial spreading malignant melanoma, acral lentiginous malignant melanoma or nodular malignant melanoma with bis(thio-hydrazide amides) represented by a formula selected from structural formulas (i)-(ix) or pharmaceutically acceptable salts thereof, pharmaceutical compositions comprising these bis(thio-hydrazide amides) and compositions comprising these bis(thiohydrazide)amides and one or more anti-cancer agent.

### RELATED APPLICATION

This application claims the benefit of U.S. Provisional Application No. 60/838,977, filed on Aug. 21, 2006. The entire teachings of the above application are incorporated herein by reference.

### BACKGROUND OF THE INVENTION

1 in 59 men and women in the US will be diagnosed with melanoma of the skin during their lifetime. 80% of melanoma cases are diagnosed while the cancer is still confined to the primary site (localized stage). 12% are diagnosed after the cancer has spread to regional lymphnodes or directly beyond the primary site. 4% are diagnosed after the cancer has already metastasized (Stage IV or metastatic melanoma). Median survival of patients with metastatic melanoma is 6 to 9 months. 7,910 men and women will die of melanoma of the skin in 2006.

Therefore, there is a great need for improvements in the methods of treatment of melanoma.

### SUMMARY OF THE INVENTION

It has now been found that certain bis(thiohydrazide) amides are effective in increasing the time to progression of the disease in melanoma compared with currently available therapies. The methods disclosed herein demonstrate a statistically significant increase in the time to progression of the disease in melanoma patients treated with compound (1) in combination with paclitaxel compared with paclitaxel alone. Disclosed are methods employing bis(thio-hydrazide amides) to treat lentigo maligna, superficial spreading malignant melanoma, acral lentiginous malignant melanoma or nodular malignant melanoma in a subject. The methods include administering to the subject an effective amount of a bis(thio-hydrazide amide) represented by Structural Formula I:

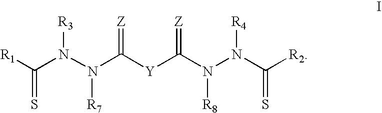

Y is a covalent bond or an optionally substituted straight chained hydrocarbyl group, or, Y, taken together with both >C=Z groups to which it is bonded, is an optionally substituted aromatic group.

R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic ring optionally fused to an aromatic ring.

R₇-R₈ are independently —H, an optionally substituted aliphatic group, or an optionally substituted aryl group.

Z is O or S;

Also disclosed are methods of preventing or delaying the recurrence of melanoma in a subject who has been treated for lentigo maligna, superficial spreading malignant melanoma, acral lentiginous malignant melanoma or nodular malignant melanoma. The methods include administering to the subject an effective amount of a bis(thio-hydrazide amide) represented by Structural Formula I.

### BRIEF DESCRIPTION OF THE DRAWINGS

FIG. 1 is a Kaplan-Meier graph of time-to-progression (resumption of cancer growth) in a study of Paclitaxel+compound (1) versus Paclitaxel alone.

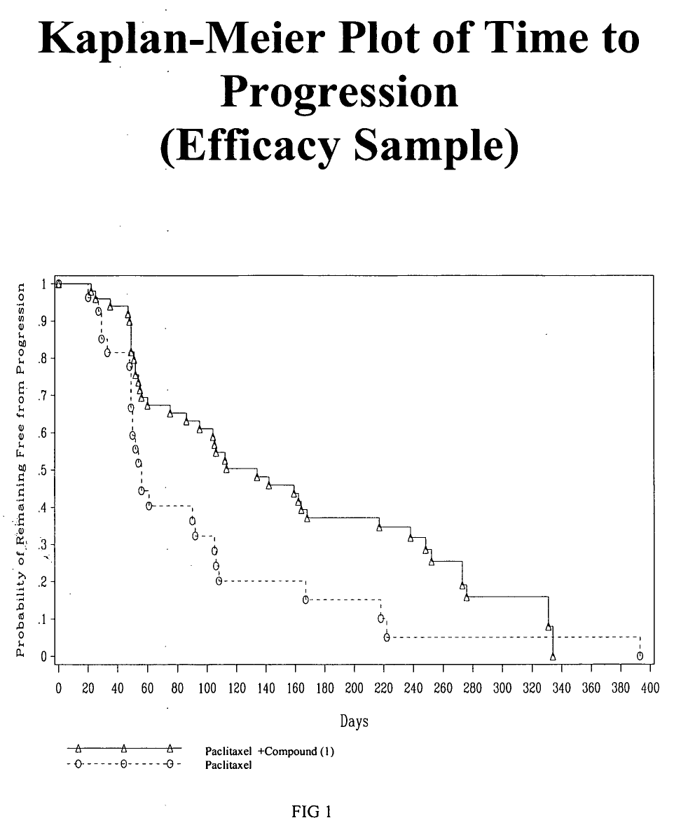

### DETAILED DESCRIPTION OF THE INVENTION

The present invention relates to methods of treating, preventing or delaying the recurrence of lentigo maligna, superficial spreading malignant melanoma, acral lentiginous malignant melanoma or nodular malignant melanoma with a bis(thio-hydrazide amide) represented by a formula selected from Structural Formulas (I)-(IX) (or a compound encompassed by these structural formulas) or a pharmaceutically acceptable salt thereof, pharmaceutical composition comprising these bis(thio-hydrazide amides) and a composition comprising these bis(thiohydrazide)amides and additional anti-cancer agents.

Yet another embodiment of the present invention is the use of a bis(thiohydrazide amide) disclosed herein for the manufacture of a medicament for treating, preventing or delaying the recurrence of lentigo maligna, superficial spreading malignant melanoma, acral lentiginous malignant melanoma or nodular malignant melanoma in a subject in need thereof.

Skin cancer begins in cells in the upper layer of skin. There are three different types of skin cancer: squamous cell carcinoma, basal cell carcinoma and melanoma.

All three types of cancer begin in the cells of the epidermis, the skin's upper layer.

Melanoma is the least common type of skin cancer, but is the most serious. It begins in the melanocytes. Melanoma is the leading cause of all skin cancer-related deaths.

Melanoma, can be divided into five main subgroups:

i) Congenital Nevus: which is congenital and not malignant.

ii) Lentigo Maligna (Hutchinsons Freckle): which is a form of melanoma more common among the elderly population. These lesions may grow for years as an in-situ tumor before developing the more aggressive vertical growth phase. This type of melanoma is found most often in the damaged skin on the face, ears, arms, and upper trunk.

iii) Superficial Spreading Malignant Melanoma: is generally the most common form accounting for approximately 65% of diagnosed melanoma. The cancer presumably begins at one focus in the skin at the dermo-epidermal junction. It initially grows in a horizontal plane, along, just above and below the dermo-epidermal junction. This is referred to as the “radial” growth phase of melanoma and is clinically macular or only slightly elevated.

This melanoma travels along the top layer of the skin for a fairly long time before penetrating more deeply. The melanoma can be seen almost anywhere on the body, but is most likely to occur on the trunk in men, the legs in women, and the upper back in both. This type of melanoma is mainly found in the younger population.

iv) Acral Lentiginous Malignant Melanoma: as with superficial spreading malignant melanoma, acral lentiginous malignant melanoma also spreads superficially before penetrating more deeply. It is quite different from the others, though, as it usually appears as a black or brown discoloration under the nails or on the soles of the feet or palms of the hands. This type of melanoma is the most common melanoma in African-Americans and Asians, and the least common among Caucasians.

v) Nodular Malignant Melanoma: is a much less common form of melanoma. Unlike the other types, nodular melanoma, is usually invasive at the time it is first diagnosed. The malignancy is recognized when it becomes a bump. In this tumor, there is presumably no horizontal growth phase. The depth of the lesion appears to correlate with the prognosis of the patient, and nodular melanoma is less often amenable to definitive treatment than is the superficial spreading variety.

The methods of the present invention encompass treating all of the subgroups of melanoma defined above.

Melanoma can further be divided into four different stages, which are divided based on the progression of the disease:

Stage I

Cancer is found in the outer layer of the skin (epidermis) and/or the upper part of the inner layer of skin (dermis), but it has not spread to nearby lymph nodes. The tumor is less than 1.5 millimeters ( 1/16 of an inch) thick.

Stage II

The tumor is 1.5 millimeters to 4 millimeters (less than ⅙ of an inch) thick. It has spread to the lower part of the inner layer of skin (dermis), but not into the tissue below the skin or into nearby lymph nodes.

Stage III

Any of the following mean that the tumor is stage III:

The tumor is more than 4 millimeters (approximately ⅙ of an inch) thick.

The tumor has spread to the body tissue below the skin.

There are additional tumor growths within one inch of the original tumor (satellite tumors).

The tumor has spread to nearby lymph nodes or there are additional tumor growths (satellite tumors) between the original tumor and the lymph nodes in the area

Stage IV

The tumor has spread to other organs or to lymph nodes far away from the original tumor.

In one embodiment, the present invention is a method of treating, preventing or delaying the recurrence of a subject with Stage I, II, III or IV Lentigo maligna comprising administering to the subject an effective amount of a bis(thiohydrazide amide) described herein.

In one embodiment, the present invention is a method of treating, preventing or delaying the recurrence of a subject with Stage I, II, III or IV superficial spreading malignant melanoma comprising administering to the subject an effective amount of a bis(thiohydrazide amide) described herein.

In one embodiment, the present invention is a method of treating, preventing or delaying the recurrence of a subject with Stage I, II, III or IV acral lentiginous malignant melanoma comprising administering to the subject an effective amount of a bis(thiohydrazide amide) described herein.

In one embodiment, the present invention is a method of treating, preventing or delaying the recurrence of a subject with Stage I, II, III or IV nodular malignant melanoma comprising administering to the subject an effective amount of a bis(thiohydrazide amide) described herein.

The bis(thio-hydrazide amides) employed in the disclosed invention are represented by Structural Formula I and pharmaceutically acceptable salts and solvates of the compounds represented by Structural Formula I.

In one embodiment, Y in Structural Formula I is a covalent bond, —C(R₅R₆)—, —(CH₂CH₂)—, trans-(CH═CH)—, cis-(CH═CH)— or —(C≡C)— group, preferably —C(R₅R₆)—. R₁-R₄ are as described above for Structural Formula I. R₅ and R₆ are each independently —H, an aliphatic or substituted aliphatic group, or R₅ is —H and R₆ is an optionally substituted aryl group, or, R₅ and R₆, taken together, are an optionally substituted C2-C6 alkylene group. In one embodiment, the compound of Structural Formula I is in the form of a pharmaceutically acceptable salt. In one embodiment, the compound of Structural Formula I is in the form of a pharmaceutically acceptable salt in combination with one or more pharmaceutically acceptable cations. The pharmaceutically acceptable cations are as described in detail below.

In specific embodiments, Y taken together with both >C=Z groups to which it is bonded, is an optionally substituted aromatic group. In this instance, certain bis(thio-hydrazide amides) are represented by Structural Formula II:

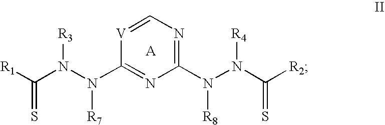

wherein Ring A is substituted or unsubstituted and V is —CH— or —N—. The other variables in Structural Formula II are as described herein for Structural Formula I or IIIa.

In particular embodiments, the bis(thio-hydrazide amides) are represented by Structural Formula IIIa:

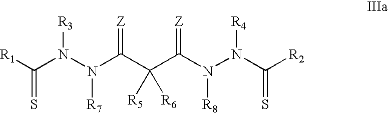

R₁-R₈ are as described above for Structural Formula I.

In Structural Formulas I-IIIa, R₁ and R₂ are the same or different and/or R₃ and R₄ are the same or different; preferably, R₁ and R₂ are the same and R₃ and R₄ are the same. In Structural Formulas I and IIIa, Z is preferably O. Typically in Structural Formulas I and IIIa, Z is O; R₁ and R₂ are the same; and R₃ and R₄ are the same. More preferably, Z is O; R₁ and R₂ are the same; R₃ and R₄ are the same, and R₇ and R₈ are the same.

In other embodiments, the bis(thio-hydrazide amides) are represented by Structural Formula IIIa: R₁ and R₂ are each an optionally substituted aryl group, preferably an optionally substituted phenyl group; R₃ and R₄ are each an optionally substituted aliphatic group, preferably an alkyl group optionally substituted with —OH, halogen, phenyl, benzyl, pyridyl, or C1-C8 alkoxy and R₆ is —H or methyl, more preferably, methyl or ethyl group optionally substituted with —OH, halogen, phenyl, benzyl, pyridyl, or C1-C8 alkoxy and R₆ is —H or methyl optionally substituted with —OH, halogen or C1-C4 alkoxy; and R₅ and R₆ are as described above, but R₅ is preferably —H and R₆ is preferably —H, an aliphatic or substituted aliphatic group.

Alternatively, R₁ and R₂ are each an optionally substituted aryl group; R₃ and R₄ are each an optionally substituted aliphatic group; R₅ is —H; and R₆ is —H, an aliphatic or substituted aliphatic group. Preferably, R₁ and R₂ are each an optionally substituted aryl group; R₃ and R₄ are each an alkyl group optionally substituted with —OH, halogen, phenyl, benzyl, pyridyl, or C1-C8 alkoxy and R₆ is —H or methyl; and R₅ is —H and R₆ is —H or methyl. Even more preferably, R₁ and R₂ are each an optionally substituted phenyl group, preferably optionally substituted with —OH, halogen, C1-4 alkyl or C1-C4 alkoxy; R₃ and R₄ are each methyl or ethyl optionally substituted with —OH, halogen or C1-C4 alkoxy; and R₅ is —H and R₆ is —H or methyl. Suitable substituents for an aryl group represented by R₁ and R₂ and an aliphatic group represented by R₃, R₄ and R₆ are as described below for aryl and aliphatic groups.

In another embodiment, the bis(thio-hydrazide amides) are represented by Structural Formula IIIa: R₁ and R₂ are each an optionally substituted aliphatic group, preferably a C3-C8 cycloalkyl group optionally substituted with at least one alkyl group, more preferably cyclopropyl or 1-methylcyclopropyl; R₃ and R₄ are as described above for Structural Formula I, preferably both an optionally substituted alkyl group; and R₅ and R₆ are as described above, but R₅ is preferably —H and R₆ is preferably —H, an aliphatic or substituted aliphatic group, more preferably —H or methyl.

Alternatively, the bis(thio-hydrazide amides) are represented by Structural Formula IIIa: R₁ and R₂ are each an optionally substituted aliphatic group; R₃ and R₄ are as described above for Structural Formula I, preferably both an optionally substituted alkyl group; and R₅ is —H and R₆ is —H or an optionally substituted aliphatic group. Preferably, R₁ and R₂ are both a C3-C8 cycloalkyl group optionally substituted with at least one alkyl group; R₃ and R₄ are both as described above for Structural Formula I, preferably an alkyl group; and R₅ is —H and R₆ is —H or an aliphatic or substituted aliphatic group. More preferably, R₁ and R₂ are both a C3-C8 cycloalkyl group optionally substituted with at least one alkyl group; R₃ and R₄ are both an alkyl group group optionally substituted with —OH, halogen, phenyl, benzyl, pyridyl, or C1-C8 alkoxy and R₆ is —H or methyl; and R₅ is —H and R₆ is —H or methyl. Even more preferably, R₁ and R₂ are both cyclopropyl or 1-methylcyclopropyl; R₃ and R₄ are both an alkyl group, preferably methyl or ethyl optionally substituted with —OH, halogen or C1-C4 alkoxy; and R₅ is —H and R₆ is —H or methyl.

In particular embodiments, the bis(thio-hydrazide amides) are represented by Structural Formula IIIb:

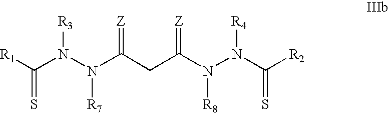

wherein R₁, R₂, R₃, R₄, R₇, R₈, and Z are as defined above for Structural Formula IIIa.

In specific embodiments, the bis(thio-hydrazide amides) are represented by Structural Formula IVa:

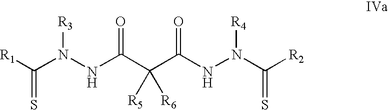

wherein: R₁ and R₂ are both phenyl, R₃ and R₄ are both methyl, and R₅ and R₆ are both —H; R₁ and R₂ are both phenyl, R₃ and R₄ are both ethyl, and R₅ and R₆ are both —H; R₁ and R₂ are both 4-cyanophenyl, R₃ and R₄ are both methyl, R₅ is methyl, and R₆ is —H; R₁ and R₂ are both 4-methoxyphenyl, R₃ and R₄ are both methyl, and R₅ and R₆ are both —H; R₁ and R₂ are both phenyl, R₃ and R₄ are both methyl, R₅ is methyl, and R₆ is —H; R₁ and R₂ are both phenyl, R₃ and R₄ are both ethyl, R₅ is methyl, and R₆ is —H; R₁ and R₂ are both 4-cyanophenyl, R₃ and R₄ are both methyl, and R₅ and R₆ are both —H; R₁ and R₂ are both 2,5-dimethoxyphenyl, R₃ and R₄ are both methyl, and R₅ and R₆ are both —H; R₁ and R₂ are both 2,5-dimethoxyphenyl, R₃ and R₄ are both methyl, R₅ is methyl, and R₆ is —H; R₁ and R₂ are both 3-cyanophenyl, R₃ and R₄ are both methyl, and R₅ and R₆ are both —H; R₁ and R₂ are both 3-fluorophenyl, R₃ and R₄ are both methyl, and R₅ and R₆ are both —H; R₁ and R₂ are both 4-chlorophenyl, R₃ and R₄ are both methyl, R₅ is methyl, and R₆ is —H; R₁ and R₂ are both 2-dimethoxyphenyl, R₃ and R₄ are both methyl, and R₅ and R₆ are both —H; R₁ and R₂ are both 3-methoxyphenyl, R₃ and R₄ are both methyl, and R₅ and R₆ are both —H; R₁ and R₂ are both 2,3-dimethoxyphenyl, R₃ and R₄ are both methyl, and R₅ and R₆ are both —H; R₁ and R₂ are both 2,3-dimethoxyphenyl, R₃ and R₄ are both methyl, R₅ is methyl, and R₆ is —H; R₁ and R₂ are both 2,5-difluorophenyl, R₃ and R₄ are both methyl, and R₅ and R₆ are both —H; R₁ and R₂ are both 2,5-difluorophenyl, R₃ and R₄ are both methyl, R₅ is methyl, and R₆ is —H; R₁ and R₂ are both 2,5-dichlorophenyl, R₃ and R₄ are both methyl, and R₅ and R₆ are both —H; R₁ and R₂ are both 2,5-dimethylphenyl, R₃ and R₄ are both methyl, and R₅ and R₆ are both —H; R₁ and R₂ are both 2,5-dimethoxyphenyl, R₃ and R₄ are both methyl, and R₅ and R₆ are both —H; R₁ and R₂ are both phenyl, R₃ and R₄ are both methyl, and R₅ and R₆ are both —H; R₁ and R₂ are both 2,5-dimethoxyphenyl, R₃ and R₄ are both methyl, R₅ is methyl, and R₆ is —H; R₁ and R₂ are both cyclopropyl, R₃ and R₄ are both methyl, and R₅ and R₆ are both —H; R₁ and R₂ are both cyclopropyl, R₃ and R₄ are both ethyl, and R₅ and R₆ are both —H; R₁ and R₂ are both cyclopropyl, R₃ and R₄ are both methyl, R₅ is methyl, and R₆ is —H; R₁ and R₂ are both 1-methylcyclopropyl, R₃ and R₄ are both methyl, and R₅ and R₆ are both —H; R₁ and R₂ are both 1-methylcyclopropyl, R₃ and R₄ are both methyl, R₅ is methyl and R₆ is —H; R₁ and R₂ are both 1-methylcyclopropyl, R₃ and R₄ are both methyl, R₅ is ethyl, and R₆ is —H; R₁ and R₂ are both 1-methylcyclopropyl, R₃ and R₄ are both methyl, R₅ is n-propyl, and R₆ is —H; R₁ and R₂ are both 1-methylcyclopropyl, R₃ and R₄ are both methyl, and R₅ and R₆ are both methyl; R₁ and R₂ are both 1-methylcyclopropyl, R₃ and R₄ are both ethyl, and R₅ and R₆ are both —H; R₁ and R₂ are both 1-methylcyclopropyl, R₃ is methyl, R₄ is ethyl, and R₅ and R₆ are both —H; R₁ and R₂ are both 2-methylcyclopropyl, R₃ and R₄ are both methyl, and R₅ and R₆ are both —H; R₁ and R₂ are both 2-phenylcyclopropyl, R₃ and R₄ are both methyl, and R₅ and R₆ are both —H; R₁ and R₂ are both 1-phenylcyclopropyl, R₃ and R₄ are both methyl, and R₅ and R₆ are both —H; R₁ and R₂ are both cyclobutyl, R₃ and R₄ are both methyl, and R₅ and R₆ are both —H; R₁ and R₂ are both cyclopentyl, R₃ and R₄ are both methyl, and R₅ and R₆ are both —H; R₁ and R₂ are both cyclohexyl, R₃ and R₄ are both methyl, and R₅ and R₆ are both —H; R₁ and R₂ are both cyclohexyl, R₃ and R₄ are both phenyl, and R₅ and R₆ are both —H; R₁ and R₂ are both methyl, R₃ and R₄ are both methyl, and R₅ and R₆ are both —H; R₁ and R₂ are both methyl, R₃ and R₄ are both t-butyl, and R₅ and R₆ are both —H; R₁ and R₂ are both methyl, R₃ and R₄ are both phenyl, and R₅ and R₆ are both —H; R₁ and R₂ are both t-butyl, R₃ and R₄ are both methyl, and R₅ and R₆ are both —H; R₁ and R₂ are ethyl, R₃ and R₄ are both methyl, and R₅ and R₆ are both —H; or R₁ and R₂ are both n-propyl, R₃ and R₄ are both methyl, and R₅ and R₆ are both —H.

In particular embodiments, the bis(thio-hydrazide amides) are represented by Structural Formula IVb:

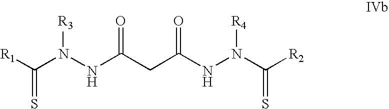

wherein R₁, R₂, R₃, and R₄ are as defined above for Structural Formula IVa.

In specific embodiments, the bis(thio-hydrazide amides) are represented by Structural Formula V:

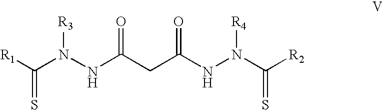

wherein: R₁ and R₂ are both phenyl, and R₃ and R₄ are both o-CH₃-phenyl; R₁ and R₂ are both o-CH₃C(O)O-phenyl, and R₃ and R₄ are phenyl; R₁ and R₂ are both phenyl, and R₃ and R₄ are both methyl; R₁ and R₂ are both phenyl, and R₃ and R₄ are both ethyl; R₁ and R₂ are both phenyl, and R₃ and R₄ are both n-propyl; R₁ and R₂ are both p-cyanophenyl, and R₃ and R₄ are both methyl; R₁ and R₂ are both p-nitro phenyl, and R₃ and R₄ are both methyl; R₁ and R₂ are both 2,5-dimethoxyphenyl, and R₃ and R₄ are both methyl; R₁ and R₂ are both phenyl, and R₃ and R₄ are both n-butyl; R₁ and R₂ are both p-chlorophenyl, and R₃ and R₄ are both methyl; R₁ and R₂ are both 3-nitrophenyl, and R₃ and R₄ are both methyl; R₁ and R₂ are both 3-cyanophenyl, and R₃ and R₄ are both methyl; R₁ and R₂ are both 3-fluorophenyl, and R₃ and R₄ are both methyl; R₁ and R₂ are both 2-furanyl, and R₃ and R₄ are both phenyl; R₁ and R₂ are both 2-methoxyphenyl, and R₃ and R₄ are both methyl; R₁ and R₂ are both 3-methoxyphenyl, and R₃ and R₄ are both methyl; R₁ and R₂ are both 2,3-dimethoxyphenyl, and R₃ and R₄ are both methyl; R₁ and R₂ are both 2-methoxy-5-chlorophenyl, and R₃ and R₄ are both ethyl; R₁ and R₂ are both 2,5-difluorophenyl, and R₃ and R₄ are both methyl; R₁ and R₂ are both 2,5-dichlorophenyl, and R₃ and R₄ are both methyl; R₁ and R₂ are both 2,5-dimethylphenyl, and R₃ and R₄ are both methyl; R₁ and R₂ are both 2-methoxy-5-chlorophenyl, and R₃ and R₄ are both methyl; R₁ and R₂ are both 3,6-dimethoxyphenyl, and R₃ and R₄ are both methyl; R₁ and R₂ are both phenyl, and R₃ and R₄ are both 2-ethylphenyl; R₁ and R₂ are both 2-methyl-5-pyridyl, and R₃ and R₄ are both methyl; or R₁ is phenyl; R₂ is 2,5-dimethoxyphenyl, and R₃ and R₄ are both methyl; R₁ and R₂ are both methyl, and R₃ and R₄ are both p-CF₃-phenyl; R₁ and R₂ are both methyl, and R₃ and R₄ are both o-CH₃-phenyl; R₁ and R₂ are both —(CH₂)₃COOH; and R₃ and R₄ are both phenyl; R₁ and R₂ are both represented by the following structural formula:

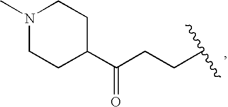

and R₃ and R₄ are both phenyl; R₁ and R₂ are both n-butyl, and R₃ and R₄ are both phenyl; R₁ and R₂ are both n-pentyl, R₃ and R₄ are both phenyl; R₁ and R₂ are both methyl, and R₃ and R₄ are both 2-pyridyl; R₁ and R₂ are both cyclohexyl, and R₃ and R₄ are both phenyl; R₁ and R₂ are both methyl, and R₃ and R₄ are both 2-ethylphenyl; R₁ and R₂ are both methyl, and R₃ and R₄ are both 2,6-dichlorophenyl; R₁-R₄ are all methyl; R₁ and R₂ are both methyl, and R₃ and R₄ are both t-butyl; R₁ and R₂ are both ethyl, and R₃ and R₄ are both methyl; R₁ and R₂ are both t-butyl, and R₃ and R₄ are both methyl; R₁ and R₂ are both cyclopropyl, and R₃ and R₄ are both methyl; R₁ and R₂ are both cyclopropyl, and R₃ and R₄ are both ethyl; R₁ and R₂ are both 1-methylcyclopropyl, and R₃ and R₄ are both methyl; R₁ and R₂ are both 2-methylcyclopropyl, and R₃ and R₄ are both methyl; R₁ and R₂ are both 1-phenylcyclopropyl, and R₃ and R₄ are both methyl; R₁ and R₂ are both 2-phenylcyclopropyl, and R₃ and R₄ are both methyl; R₁ and R₂ are both cyclobutyl, and R₃ and R₄ are both methyl; R₁ and R₂ are both cyclopentyl, and R₃ and R₄ are both methyl; R₁ is cyclopropyl, R₂ is phenyl, and R₃ and R₄ are both methyl.

Preferred examples of bis(thio-hydrazide amides) include Compounds (1)-(18) and pharmaceutically acceptable salts and solvates thereof:

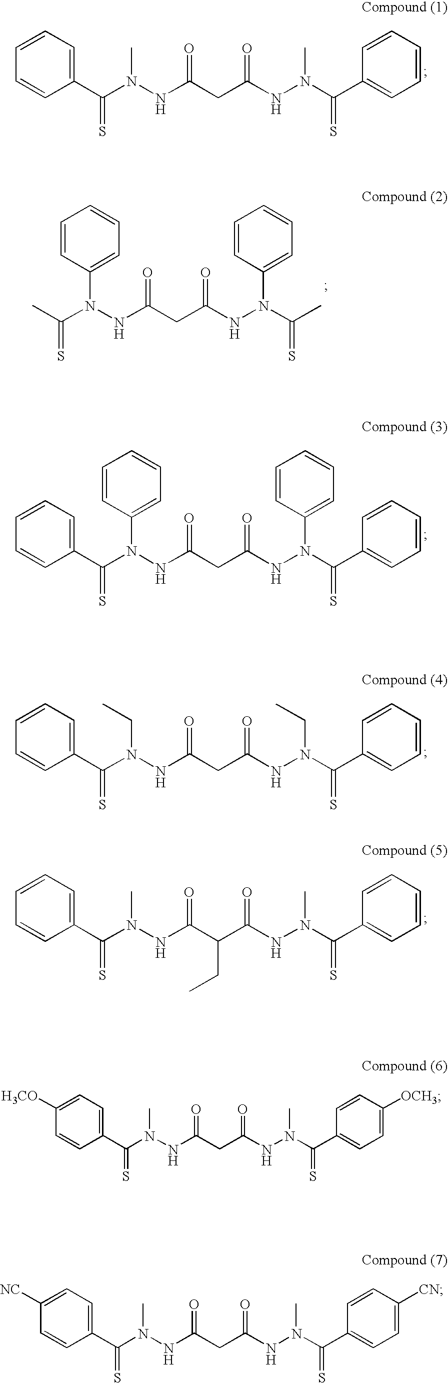

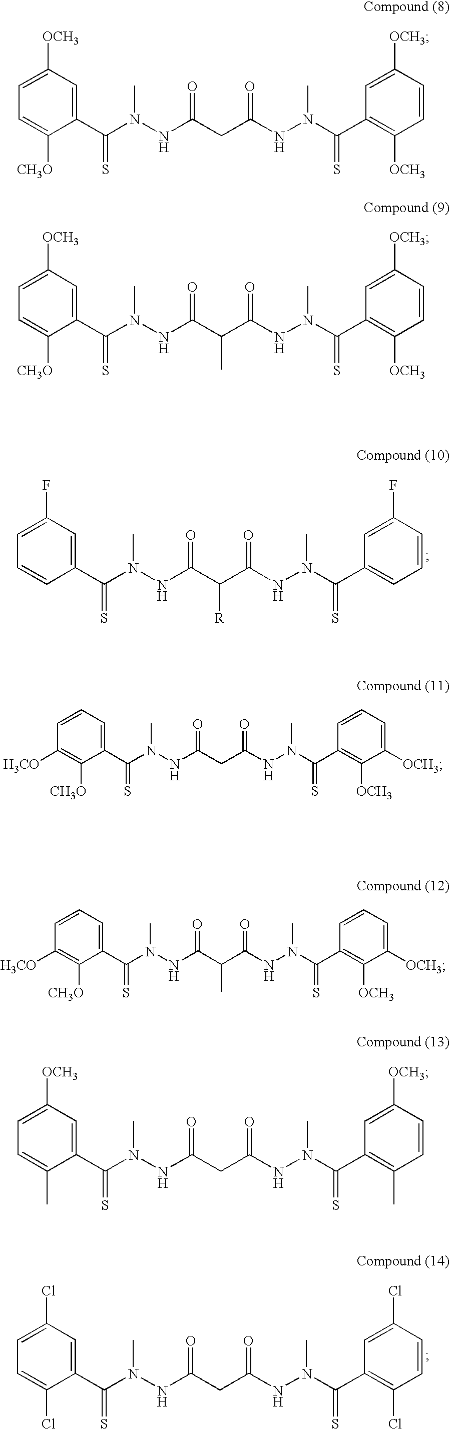

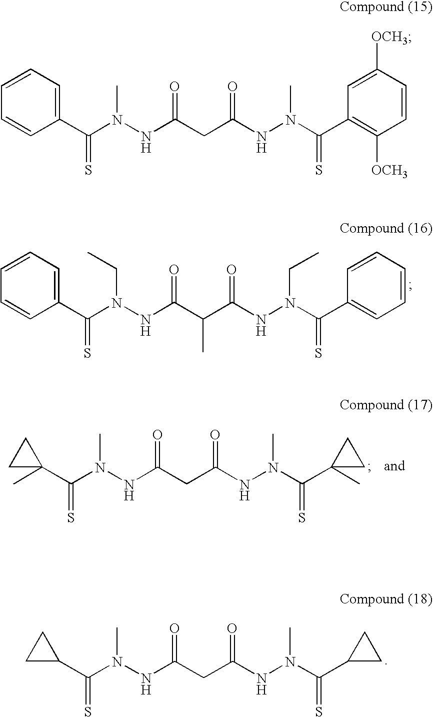

As used herein, the term “bis(thio-hydrazide amide)” and references to the Structural Formulas of this invention also include pharmaceutically acceptable salts and solvates of these compounds and Structural Formulas. Examples of acceptable salts and solvates are described in US Publication No.: 20060135595 and U.S. patent application Ser. No. 11/432,307 filed 11 May 2006, titled Synthesis Of Bis(Thio-Hydrazide Amide) Salts, the entire contents of each of which are incorporated herein by reference.

These compounds can have one or more sufficiently acidic proton that can react with a suitable organic or inorganic base to form a base addition salt. Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases useful in preparing the salts of this invention thus include sodium hydroxide, potassium hydroxide, ammonium hydroxide, potassium carbonate, and the like.

For example, pharmaceutically acceptable salts of bis(thio-hydrazide) amides employed herein (e.g., those represented by Structural Formulas I-VI, Compounds 1-18) are those formed by the reaction of the compound with one equivalent of a suitable base to form a monovalent salt (i.e., the compound has single negative charge that is balanced by a pharmaceutically acceptable counter cation, e.g., a monovalent cation) or with two equivalents of a suitable base to form a divalent salt (e.g., the compound has a two-electron negative charge that is balanced by two pharmaceutically acceptable counter cations, e.g., two pharmaceutically acceptable monovalent cations or a single pharmaceutically acceptable divalent cation). Divalent salts of the bis(thio-hydrazide amides) are preferred. “Pharmaceutically acceptable” means that the cation is suitable for administration to a subject. Examples include Li⁺, Na⁺, K⁺, Mg²⁺, Ca²⁺ and NR₄⁺, wherein each R is independently hydrogen, an optionally substituted aliphatic group (e.g., a hydroxyalkyl group, aminoalkyl group or ammoniumalkyl group) or optionally substituted aryl group, or two R groups, taken together, form an optionally substituted non-aromatic heterocyclic ring optionally fused to an aromatic ring. Generally, the pharmaceutically acceptable cation is Li⁺, Na⁺, K⁺, NH₃(C2H₅OH)⁺ or N(CH₃)₃(C2H₅OH)⁺, and more typically, the salt is a disodium or dipotassium salt, preferably the disodium salt.

Bis(thio-hydrazide) amides employed herein having a sufficiently basic group, such as an amine can react with an organic or inorganic acid to form an acid addition salt. Acids commonly employed to form acid addition salts from compounds with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Salts of the disclosed bis(thiohydrazide amides) may have tautomeric forms. By way of example, one tautomeric form for the disalt is:

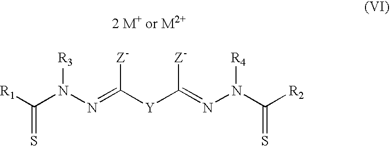

Y is a covalent bond or a substituted or unsubstituted straight chained hydrocarbyl group. R₁-R₄ are independently —H, an aliphatic group, a substituted aliphatic group, an aryl group or a substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring. Z is —O or —S. M⁺ is a pharmaceutically acceptable monovalent cation and M²⁺ is a pharmaceutically acceptable divalent cation.

In one embodiment, the variables for Structural Formula (VI) are defined below:

M⁺ is a pharmaceutically acceptable monovalent cation. M²⁺ is a pharmaceutically acceptable divalent cation. “Pharmaceutically acceptable” means that the cation is suitable for administration to a subject. Examples of M+ or M²⁺ include Li⁺, Na⁺, K⁺, Mg²⁺, Ca²⁺, Zn²⁺, and NR⁴⁺, wherein each R is independently hydrogen, a substituted or unsubstituted aliphatic group (e.g., a hydroxyalkyl group, aminoalkyl group or ammoniumalkyl group) or substituted or unsubstituted aryl group, or two R groups, taken together, form a substituted or unsubstituted non-aromatic heterocyclic ring optionally fused to an aromatic ring. Preferably, the pharmaceutically acceptable cation is Li⁺, Na⁺, K⁺, NH₃(C2H₅OH)⁺, N(CH₃)₃(C2H₅OH)⁺, arginine or lysine. More preferably, the pharmaceutically acceptable cation is Na⁺ or K⁺. Na⁺ is even more preferred.

Exemplary tautomeric forms of the disalt compounds represented by Structural Formula (VI) wherein Y is —CH₂— are shown below:

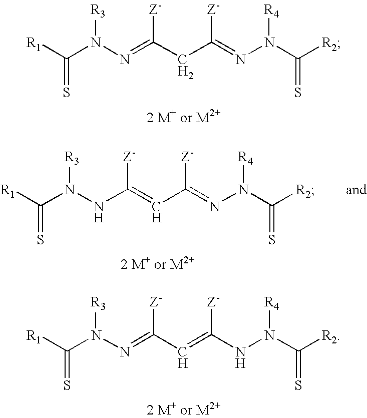

Representative tautomeric structures of the disalt of Compound (1) are shown below:

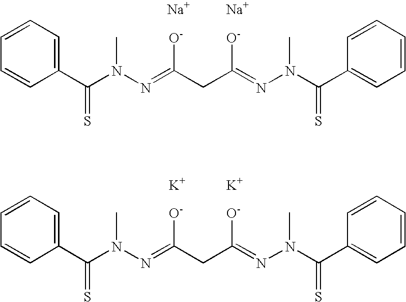

Preferred examples of bis(thio-hydrazide amide) disalts of the present invention are the following:

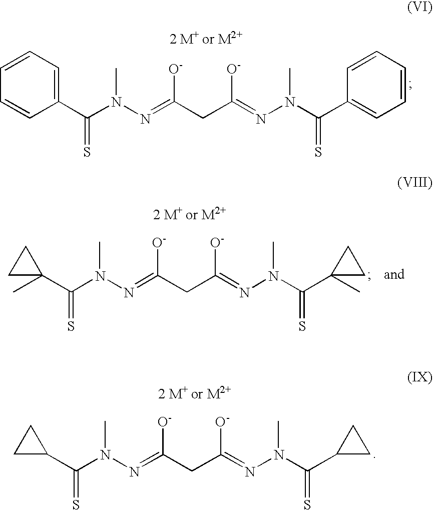

2 M⁺ and M²⁺ are as described above for Structural Formula (VI). Preferably, the pharmaceutically acceptable cation is 2 M⁺, wherein M⁺ is Li⁺, Na⁺, K⁺, NH₃(C2H₅OH)⁺ or N(CH₃)₃(C2H₅OH)⁺. More preferably, M⁺ is Na⁺ or K⁺. Even more preferably, M⁺ is Na⁺.

It is to be understood when one tautomeric form of a disclosed compound is depicted structurally, other tautomeric forms are also encompassed.

Certain compounds of the invention may be obtained as different stereoisomers (e.g., diastereomers and enantiomers). The invention includes all isomeric forms and racemic mixtures of the disclosed compounds and methods of treating a subject with both pure isomers and mixtures thereof, including racemic mixtures. Stereoisomers can be separated and isolated using any suitable method, such as chromatography.

An “alkyl group” is saturated straight or branched chain linear or cyclic hydrocarbon group. Typically, a straight chained or branched alkyl group has from 1 to about 20 carbon atoms, preferably from 1 to about 10, and a cyclic alkyl group has from 3 to about 10 carbon atoms, preferably from 3 to about 8. An alkyl group is preferably a straight chained or branched alkyl group, e.g, methyl, ethyl, n-propyl, iso-propyl, n-butyl, sec-butyl, tert-butyl, pentyl, hexyl, pentyl or octyl, or a cycloalkyl group with 3 to about 8 carbon atoms. A C1-C8 straight chained or branched alkyl group or a C3-C8 cyclic alkyl group is also referred to as a “lower alkyl” group. Suitable substitutents for an alkyl group are those which do not substantially interfere with the anti-cancer activity of the disclosed compounds. Suitable substituents are as described below for aliphatic groups. Preferred substituents on alkyl groups include, —OH, —NH₂, —NO₂, —CN, —COOH, halogen, aryl, C1-C8 alkoxy, C1-C8 haloalkoxy and —CO(C1-C8 alkyl). More preferred substituents on alkyl groups include —OH, halogen, phenyl, benzyl, pyridyl, and C1-C8 alkoxy. More preferred substituents on alkyl groups include —OH, halogen, and C1-C4 alkoxy.

A “straight chained hydrocarbyl group” is an alkylene group, i.e., —(CH₂)_{y}—, with one or more (preferably one) internal methylene groups optionally replaced with a linkage group. y is a positive integer (e.g., between 1 and 10), preferably between 1 and 6 and more preferably 1 or 2. A “linkage group” refers to a functional group which replaces a methylene in a straight chained hydrocarbyl. Examples of suitable linkage groups include a ketone (—C(O)—), alkene, alkyne, phenylene, ether (—O—), thioether (—S—), or amine (—N(R^{a})—), wherein R^{a} is defined below. A preferred linkage group is —C(R₅R₆)—, wherein R₅ and R₆ are defined above. Suitable substitutents for an alkylene group and a hydrocarbyl group are those which do not substantially interfere with the anti-cancer activity of the disclosed compounds. R₅ and R₆ are preferred substituents for an alkylene or hydrocarbyl group represented by Y.

An aliphatic group is a straight chained, branched or cyclic non-aromatic hydrocarbon which is completely saturated or which contains one or more units of unsaturation. Typically, a straight chained or branched aliphatic group has from 1 to about 20 carbon atoms, preferably from 1 to about 10, and a cyclic aliphatic group has from 3 to about 10 carbon atoms, preferably from 3 to about 8. An aliphatic group is preferably a straight chained or branched alkyl group, e.g, methyl, ethyl, n-propyl, iso-propyl, n-butyl, sec-butyl, tert-butyl, pentyl, hexyl, pentyl or octyl, or a cycloalkyl group with 3 to about 8 carbon atoms. A C1-C8 straight chained or branched alkyl group or a C3-C8 cyclic alkyl group is also referred to as a “lower alkyl” group.

The term “aromatic group” may be used interchangeably with “aryl,” “aryl ring,” “aromatic ring,” “aryl group” and “aromatic group.” Aromatic groups include carbocyclic aromatic groups such as phenyl, naphthyl, and anthracyl, and heteroaryl groups such as imidazolyl, thienyl, furanyl, pyridyl, pyrimidy, pyranyl, pyrazolyl, pyrroyl, pyrazinyl, thiazole, oxazolyl, and tetrazole. The term “heteroaryl group” may be used interchangeably with “heteroaryl,” “heteroaryl ring,” “heteroaromatic ring” and “heteroaromatic group.” Heteroaryl groups are aromatic groups that comprise one or more heteroatom, such as sulfur, oxygen and nitrogen, in the ring structure. Preferably, heteroaryl groups comprise from one to four heteroatoms.

Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

Non-aromatic heterocyclic rings are non-aromatic rings which include one or more heteroatoms such as nitrogen, oxygen or sulfur in the ring. The ring can be five, six, seven or eight-membered. Preferably, heterocyclic groups comprise from one to about four heteroatoms. Examples include tetrahydrofuranyl, tetrahyrothiophenyl, morpholino, thiomorpholino, pyrrolidinyl, piperazinyl, piperidinyl, and thiazolidinyl.

Suitable substituents on an aliphatic group (including an alkylene group), non-aromatic heterocyclic group, benzylic or aryl group (carbocyclic and heteroaryl) are those which do not substantially interfere with the anti-cancer activity of the disclosed compounds. A substituent substantially interferes with anti-cancer activity when the anti-cancer activity is reduced by more than about 50% in a compound with the substituent compared with a compound without the substituent. Examples of suitable substituents include —R^{a}, —OH, —Br, —Cl, —I, —F, —OR^{a}, —O—COR^{a}, —COR^{a}, —CN, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCCOR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR —C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR^{a}, —SO₂NR^{a}R^{b}, —CH═CHR^{a}, —CH═CR^{a}R^{b}, CR^{c}═CR^{a}R^{b}, CR^{c}=CHR^{a}, —CR^{c}=CR^{a}R^{b}, —CCR^{a}, —SH, —SR^{a}, —S(O)R^{a}, —S(O)₂R^{a}.

R^{a}-R^{d} are each independently an alkyl group, aromatic group, non-aromatic heterocyclic group or —N(R^{a}R^{b}), taken together, form a non-aromatic heterocyclic group. The alkyl, aromatic and non-aromatic heterocyclic group represented by R^{a}-R^{d} and the non-aromatic heterocyclic group represented by —N(R^{a}R^{b}) are each optionally and independently substituted with one or more groups represented by R#. Preferably R^{a}-R^{d} are unsubstituted.

R# is R⁺, —OR⁺, —O(haloalkyl), —SR⁺, —NO₂, —CN, —NCS, —N(R⁺)₂, —NHCO₂R⁺, —NHC(O)R⁺, —NHNHC(O)R⁺, —NHC(O)N(R⁺)₂, —NHNHC(O)N(R⁺)₂, —NHNHCO₂R⁺, —C(O)C(O)R⁺, —C(O)CH₂C(O)R⁺, —CO₂R⁺, —C(O)R⁺, —C(O)N(R⁺)₂, —OC(O)R⁺, —OC(O)N(R⁺)₂, —S(O)₂R⁺, —SO₂N(R⁺)₂, —S(O)R⁺, —NHSO₂N(R⁺)₂, —NHSO₂R⁺, —C(═S)N(R⁺)₂, or —C(═NH)—N(R⁺)₂.

R⁺ is —H, a C1-C4 alkyl group, a monocyclic heteroaryl group, a non-aromatic heterocyclic group or a phenyl group optionally substituted with alkyl, haloalkyl, alkoxy, haloalkoxy, halo, —CN, —NO₂, amine, alkylamine or dialkylamine. Preferably R⁺ is unsubstituted. Optionally, the group —N(R⁺)₂ is a non-aromatic heterocyclic group, provided that non-aromatic heterocyclic groups represented by R⁺ and —N(R⁺)₂ that comprise a secondary ring amine are optionally acylated or alkylated. Preferred substituents for a phenyl group, including phenyl groups represented by R₁-R₄, include C1-C4 alkyl, C1-C4 alkoxy, C1-C4 haloalkyl, C1-C4 haloalkoxy, phenyl, benzyl, pyridyl, —OH, —NH₂, —F, —Cl, —Br, —I, —NO₂ or —CN. More preferred for a phenyl group, including phenyl groups represented by R₁-R₄, include R₁ and R₂ are optionally substituted with —OH, —CN, halogen, C1-4 alkyl or C1-C4 alkoxy

Preferred substituents for a cycloalkyl group, including cycloalkyl groups represented by R₁ and R₂, are alkyl groups, such as a methyl or ethyl group.

In one embodiment of the present invention the bis(thiohydrazide amides) described herein can be administered to a subject in the form of a pharmaceutical composition.

As used herein, a “pharmaceutical composition” can be a formulation containing the disclosed compounds, in a form suitable for administration to a subject. The pharmaceutical composition can be in bulk or in unit dosage form. The unit dosage form can be in any of a variety of forms, including, for example, a capsule, an IV bag, a tablet, a single pump on an aerosol inhaler, or a vial. The quantity of active ingredient (i.e., a formulation of the disclosed compound or salts thereof) in a unit dose of composition can be an effective amount and can be varied according to the particular treatment involved. It may be appreciated that it can be necessary to make routine variations to the dosage depending on the age and condition of the patient. The dosage can also depend on the route of administration. Examples of suitable dosages are those described in PCT/US2006/014531 filed 13 Apr. 2006, titled Combination Cancer Therapy With Bis[Thiohydrazide] Amide Compounds, the entire contents of which are incorporated herein by reference. A variety of routes are contemplated, including topical, oral, pulmonary, rectal, vaginal, parenternal, including transdermal, subcutaneous, intravenous, intramuscular, intraperitoneal and intranasal.

The compounds described herein, and the pharmaceutically acceptable salts thereof can be used in pharmaceutical preparations in combination with a pharmaceutically acceptable carrier or diluent. Suitable pharmaceutically acceptable carriers include inert solid fillers or diluents and sterile aqueous or organic solutions. The compounds can be present in such pharmaceutical compositions in amounts sufficient to provide the desired dosage amount in the range described herein. Techniques for formulation and administration of the disclosed compounds of the invention can be found in Remington: the Science and Practice of Pharmacy, 19^{th} edition, Mack Publishing Co., Easton, Pa. (1995). The bis(thio-hydrazide amide) disclosed herein can be prepared by the methods described in U.S. Provisional Patent No. 60/708,977 filed 16 Aug. 2005, titled Bis(Thio-Hydrazide Amide) Formulation, the entire teachings of which is incorporated herein by reference.

In one embodiment the bis(thio hydrazide amide) described herein is added to a solution of paclitaxel in Cremophor®. In one embodiment, paclitaxel is 6 mg/mL and the bis(thiohydrazid amide) (e.g., compound (1) is 16 mg/L in the Cremophor® solution. Optionally, the solution is then diluted with a saline solution Specifically, for Intravenous Administration: paclitaxel is diluted prior to infusion, for example, paclitaxel is diluted in 0.9% Sodium Chloride Injection, USP; 5% Dextrose Injection, USP; 5% Dextrose and 0.9% Sodium Chloride Injection, USP, or 5% Dextrose in Ringer's Injection to a final concentration of 0.3 to 1.2 mg/mL.

For oral administration, the disclosed compounds or salts thereof can be combined with a suitable solid or liquid carrier or diluent to form capsules, tablets, pills, powders, syrups, solutions, suspensions, or the like.

The tablets, pills, capsules, and the like can contain from about 1 to about 99 weight percent of the active ingredient and a binder such as gum tragacanth, acacias, corn starch or gelatin; excipients such as dicalcium phosphate; a disintegrating agent such as corn starch, potato starch or alginic acid; a lubricant such as magnesium stearate; and/or a sweetening agent such as sucrose, lactose or saccharin. When a dosage unit form is a capsule, it may contain, in addition to materials of the above type, a liquid carrier such as a fatty oil.

Various other materials can be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like.

For parental administration, the bis(thio-hydrazide) amides can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition to the formulations previously described, the compounds may also be formulated as a depot preparation. Suitable formulations of this type include biocompatible and biodegradable polymeric hydrogel formulations using crosslinked or water insoluble polysaccharide formulations, polymerizable polyethylene oxide formulations, impregnated membranes, and the like. Such long acting formulations may be administered by implantation or transcutaneous delivery (for example subcutaneously or intramuscularly), intramuscular injection or a transdermal patch. Typically, they can be implanted in, or applied to, the microenvironment of an affected organ or tissue, for example, a membrane impregnated with the disclosed compound can be applied to an open wound or burn injury. Thus, for example, the compounds may be formulated with suitable polymeric or hydrophobic materials, for example, as an emulsion in an acceptable oil, or ion exchange resins, or as sparingly soluble derivatives, for example, as a sparingly soluble salt.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, a cream formulation can be administer to a wound site, or a bandage may be impregnated with a formulation, and the like.

For rectal administration, suitable pharmaceutical compositions are, for example, topical preparations, suppositories or enemas.

For vaginal administration, suitable pharmaceutical compositions are, for example, topical preparations, pessaries, tampons, creams, gels, pastes, foams or sprays.

In addition, the compounds may also be formulated to deliver the active agent by pulmonary administration, e.g., administration of an aerosol formulation containing the active agent from, for example, a manual pump spray, nebulizer or pressurized metered-dose inhaler. Suitable formulations of this type can also include other agents, such as antistatic agents, to maintain the disclosed compounds as effective aerosols.

The term “pulmonary” as used herein refers to any part, tissue or organ whose primary function is gas exchange with the external environment, i.e., O₂/CO₂ exchange, within a patient. “Pulmonary” typically refers to the tissues of the respiratory tract. Thus, the phrase “pulmonary administration” refers to administering the formulations described herein to any part, tissue or organ whose primary function is gas exchange with the external environment (e.g., mouth, nose, pharynx, oropharynx, laryngopharynx, larynx, trachea, carina, bronchi, bronchioles, alveoli). For purposes of the present invention, “pulmonary” is also meant to include a tissue or cavity that is contingent to the respiratory tract, in particular, the sinuses.

A drug delivery device for delivering aerosols can comprise a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

For nasal administration, either a solid or a liquid carrier can be used. The solid carrier includes a coarse powder having particle size in the range of, for example, from about 20 to about 500 microns and such formulation is administered by rapid inhalation through the nasal passages. Where the liquid carrier is used, the formulation may be administered as a nasal spray or drops and may include oil or aqueous solutions of the active ingredients.

In addition to the formulations described above, a formulation can optionally include, or be co-administered with one or more additional drugs. The formulation may also contain preserving agents, solubilizing agents, chemical buffers, surfactants, emulsifiers, colorants, odorants and sweeteners.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

As noted above, one embodiment of the present invention is directed to treating subjects with lentigo maligna, superficial spreading malignant melanoma, acral lentiginous malignant melanoma or nodular malignant melanoma. “Treating a subject with lentigo maligna, superficial spreading malignant melanoma, acral lentiginous malignant melanoma or nodular malignant melanoma” includes achieving, partially or substantially, one or more of the following results: arresting the growth or spread of the cancer, reducing the extent of the cancer (e.g., reducing size of a tumor or reducing the number of affected sites), inhibiting the growth rate of the cancer, and ameliorating or improving a clinical symptom or indicator associated with the cancer. “Treating a subject with lentigo maligna, superficial spreading malignant melanoma, acral lentiginous malignant melanoma or nodular malignant melanoma” also includes partially or totally inhibiting, delaying or preventing the progression of cancer including cancer metastasis; partially or totally inhibiting, delaying or preventing the recurrence of cancer including cancer metastasis; or partially or totally preventing the onset or development of cancer (chemoprevention). Partially or totally inhibiting, delaying or preventing the recurrence of means inhibiting, delaying or preventing the recurrence of the cancer, after the original tumor has been removed, for example, by surgery. A subject who has been “treated for lentigo maligna, superficial spreading malignant melanoma, acral lentiginous malignant melanoma or nodular malignant melanoma”, is a subject in which the primary tumor in lentigo maligna, superficial spreading malignant melanoma, acral lentiginous malignant melanoma or nodular malignant melanoma has been removed, for example, surgically.

The term “effective amount” is the quantity of compound in which a beneficial clinical outcome is achieved when the compound is administered to a subject with a cancer. A “beneficial clinical outcome” includes prevention, inhibition or a delay in the recurrence of cancer, a reduction in tumor mass, a reduction in metastasis, a reduction in the severity of the symptoms associated with the cancer and/or an increase in the longevity of the subject compared with the absence of the treatment. The precise amount of compound (or other anti-cancer agent) administered to a subject will depend on the type and severity of the disease or condition and on the characteristics of the subject, such as general health, age, sex, body weight and tolerance to drugs. It will also depend on the degree, severity and type of cancer. The skilled artisan will be able to determine appropriate dosages depending on these and other factors. Effective amounts of the disclosed compounds typically range between about 1 mg/mm² per day and about 10 grams/mm² per day, and preferably between 10 mg/mm² per day and about 5 grams/mm². When co-administered with another anti-cancer agent, an “effective amount” of the second anti-cancer agent will depend on the type of drug used. Suitable dosages are known for approved anti-cancer agents and can be adjusted by the skilled artisan according to the condition of the subject, the type of cancer being treated and the amount of bis(thio-hydrazide amide) disalt being used.

Examples of specific dosage regimens for the disclosed compounds used in combination with taxanes are provided below. When combined with an immunotherapy, it is understood that an effective amount of the immunotherapy is also used

One dosage regimen includes the step of co-administering to the subject over three to five weeks, a taxane in an amount of between about 243 μmol/m2 to 315 μmol/m2 (e.g., equivalent to paclitaxel in about 210-270 mg/m2); and a bis(thiohydrazide amide) (e.g., as represented by Structural Formula I) in an amount between about 1473 μmol/m2 and about 1722 μmol/m2 (e.g., Compound (1) in about 590-690 mg/m2).

In another dosage regimen the taxane and the bis(thio-hydrazide) amide can each be administered in three equal weekly doses for three weeks of a four week period. In preferred embodiments, the four week administration period can be repeated until the cancer is in remission. The taxane can be any taxane defined herein. In a specific embodiment, the taxane is paclitaxel intravenously administered in a weekly dose of about 94 μmol/m2 (80 mg/m2). Typically, the bis(thiohydrazide amide) can be intravenously administered in a weekly dose of between about 500 μmol/m2 and about 562 μmol/m2, or more typically in a weekly dose of about 532 μmol/m2. (e.g., Compound (1) in about 590-690 mg/m2).

Another dosage regimen includes intravenously administering to the subject in a four week period, three equal weekly doses of paclitaxel in an amount of about 94 μmol/m2; and compound (1) or a pharmaceutically acceptable salt or solvate thereof in an amount of about 532 μmol/m2.

In another dosage regimen, the subject can be intravenously administered between about 220 μmol/m2 and about 1310 μmol/m2 (e.g., Compound (1) in about 88-525 mg/m2) of the bis(thiohydrazide amide) once every 3 weeks, generally between about 220 μmol/m2 and about 1093 μmol/m2 (e.g., Compound (1) in about 88-438 mg/m2) once every 3 weeks, typically between about 624 μmol/m2 and about 1124 μmol/m2 m2 (e.g., Compound (1) in about 250-450 mg/m2), more typically between about 811 μmol/m2 and about 936 μmol/m2 m2 (e.g., Compound (1) in about 325-375 mg/m2), or in particular embodiments, about 874 μmol/m2 ((e.g., Compound (1) in about 350 mg/m2). In particular embodiments, the subject can be intravenously administered between about 582 μmol/m2 and about 664 μmol/m2 (e.g., Compound (1) in about 233-266 mg/m2) of the bis(thiohydrazide amide) once every 3 weeks. In certain embodiments, the bis(thiohydrazide amide) is in an amount of about 664 μmol/m2 (e.g., Compound (1) in about 266 mg/m2).

In another dosage regimen, the subject can be intravenously administered between about 200 μmol/m2 to about 263 μmol/m2 of the taxane as paclitaxel once every 3 weeks (e.g., paclitaxel in about 175-225 mg/m2). In some embodiments, the subject can be intravenously administered between about 200 μmol/m2 to about 234 μmol/m2 of the taxane as paclitaxel once every 3 weeks (e.g., paclitaxel in about 175-200 mg/m2). In certain embodiments, the paclitaxel is administered in an amount of about 234 μmol/m2 (200 mg/m2). In certain embodiments, the paclitaxel is administered in an amount of about 205 μmol/m2 (175 mg/m2).

In one embodiment, the taxane, e.g., paclitaxel, and the bis(thiohydrazide amide), e.g., Compound (1), can be administered together in a single pharmaceutical composition.

In one embodiment, the method of the present invention includes treating a subject once every three weeks, independently or together a taxane in an amount of about 205 μmol/m2 (e.g., paclitaxel in about 175 mg/m2); and a bis(thiohydrazide amide) represented by Structural Formula I or a pharmaceutically acceptable salt or solvate thereof in an amount between about 220 μmol/m2 and about 1310 μmol/m2 (e.g., Compound (1) in about 88-525 mg/m2). Typically, the taxane is paclitaxel intravenously administered in an amount of about 205 μmol/m2. The bis(thiohydrazide amide) can typically be intravenously administered between about 220 μmol/m2 and about 1093 μmol/m2 (e.g., Compound (1) in about 88-438 mg/m2), more typically between about 749 μmol/m2 and about 999 μmol/m2 (e.g., compound (1) in about 300-400 mg/m2), in some embodiments between about 811 mmol/m2 and about 936 μmol/m2 (e.g., Compound (1) in about 325-375 mg/m2). In certain embodiments, the bis(thiohydrazide amide) can be Compound (1) intravenously administered between about 874 μmol/m2 (about 350 mg/m2).

In a particular embodiment, the methods of the present invention involve intravenously administering to the subject in a single dose per three week period: paclitaxel in an amount of about 205 μmol/m2 (175 mg/m2); and Compound (1) or a pharmaceutically acceptable salt or solvate thereof in an amount of about 874 μmol/m2 (350 mg/m2).

Particular formulations, dosages and modes of administration are as described in US Publication No. 20060135595 and PCT/US2006/014531 filed 13 Apr. 2006, titled Combination Cancer Therapy With Bis[Thiohydrazide] Amide Compounds the entire contents of each of which are incorporated herein by reference)

The bisthiohydrazide amide) can be administered in combination with an effective amount of an anti-cancer therapy selected from: anti-cancer agents/drugs, biological therapy (e.g., immunotherapy drugs), radiation therapy, anti-angiogenesis therapy, gene therapy or hormonal therapy.

In one embodiment, the present invention is a method of treating a subject with lentigo maligna, superficial spreading malignant melanoma, acral lentiginous malignant melanoma or nodular malignant melanoma, comprising administering an effective amount one or more additional anti-cancer drugs with bis(thio-hydrazide amide). Examples of anti-cancer drugs are described below. Preferably, the co-administered anti-cancer drug is an agent that stabilizes mictotubules, such as paclitaxel or an analog of paclitaxel.

In one embodiment the anti-cancer agents/drug is, for example, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents/drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpini; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

Examples of therapeutic antibodies that can be used include but are not limited to HERCEPTIN® (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/Medlmmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatied anti-CD80 antibody (IDEC Pharm/Mitsubishi); ZEVALN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CD20-sreptdavidin (+biotin-yttrium 90; NeoRx); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

Agents that can be used in the methods of the invention in combination with the bis(thiohydrazide amides) disclosed herein, include but are not limited to, alkylating agents, antimetabolites, natural products, or hormones. Examples of alkylating agents useful in the methods of the invention include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites useful in the methods of the invention include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of natural products useful in the methods of the invention include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin), enzymes (e.g., L-asparaginase), or biological response modifiers (e.g., interferon alpha). Examples of hormones and antagonists useful for the treatment or prevention of cancer in the methods and compositions of the invention include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and with the compositions of the invention for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, amihoglutethimide).

In one embodiment, microtubulin stabilizers can be used in the methods of the invention in combination with the bis(thiohydrazide amides) disclosed herein. As used herein, a “microtubulin stabilizer” means an anti-cancer agent/drug which acts by arresting cells in the G2-M phases due to stabilization of microtubules. Examples of microtubulin stabilizers include ACLITAXEL® and paclitaxel analogues. Additional examples of microtubulin stabilizers included without limitation the following marketed drugs and drugs in development: Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof.

As used herein, a “microtubulin inhibitor” means an anti-cancer agent which acts by inhibiting tubulin polymerization or microtubule assembly. Examples of microtubulin inhibitors include without limitation the following marketed drugs and drugs in development: Erbulozole (also known as R-55104); Dolastatin 10 (also known as DLS-10 and NSC-376128); Mivobulin isethionate (also known as CI-980); Vincristine; NSC-639829; ABT-751 (Abbot, also known as E-7010); Altorhyrtins (such as Altorhyrtin A and Altorhyrtin C); Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9); Cemadotin hydrochloride (also known as LU-103793 and NSC-D-669356); Auristatin PE (also known as NSC-654663); Soblidotin (also known as TZT-1027), LS-4559-P (Pharmacia, also known as LS-4577); LS-4578 (Pharmacia, also known as LS-477-P); LS-4477 (Pharmacia), LS-4559 (Pharmacia); RPR-112378 (Aventis); Vincristine sulfate; DZ-3358 (Daiichi); GS-164 (Takeda); GS-198 (Takeda); KAR-2 (Hungarian Academy of Sciences); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (also known as LY-355703); Vitilevuamide; Tubulysin A; Canadensol; Centaureidin (also known as NSC-106969); T-138067 (Tularik, also known as T-67, TL-138067 and TI-138067); COBRA-1 (Parker Hughes Institute, also known as DDE-261 and WHI-261); H10 (Kansas State University); H16 (Kansas State University); Oncocidin A1 (also known as BTO-956 and DIME); DDE-313 (Parker Hughes Institute); SPA-2 (Parker Hughes Institute); SPA-1 (Parker Hughes Institute, also known as SPIKET-P); 3-IAABU (Cytoskeleton/Mt. Sinai School of Medicine, also known as MF-569); Narcosine (also known as NSC-5366); Nascapine, D-24851 (Asta Medica), A-105972 (Abbott); Hemiasterlin; 3-BAABU (Cytoskeleton/Mt. Sinai School of Medicine, also known as MF-191); TMPN (Arizona State University); Vanadocene acetylacetonate; T-138026 (Tularik); Monsatrol; Inanocine (also known as NSC-698666); 3-IAABE (Cytoskeleton/Mt. Sinai School of Medicine); A-204197 (Abbott); T-607 (Tularik, also known as T-900607); RPR-115781 (Aventis); Eleutherobins (such as Desmethyleleutherobin, Desaetyleleutherobin, Isoeleutherobin A, and Z-Eleutherobin); Halichondrin B; D-64131 (Asta Medica); D-68144 (Asta Medica); Diazonamide A; A-293620 (Abbott); NPI-2350 (Nereus); TUB-245 (Aventis); A-259754 (Abbott); Diozostatin; (−)-Phenylahistin (also known as NSCL-96F037); D-68838 (Asta Medica); D-68836 (Asta Medica); Myoseverin B; D-43411 (Zentaris, also known as D-81862); A-289099 (Abbott); A-318315 (Abbott); HTI-286 (also known as SPA-110, trifluoroacetate salt) (Wyeth); D-82317 (Zentaris); D-82318 (Zentaris); SC-12983 (NCI); Resverastatin phosphate sodium; BPR-0Y-007 (National Health Research Institutes); SSR-250411 (Sanofi); Combretastatin A4; and analogs and derivatives thereof.

Paclitaxel, also referred to as “Taxol®”, is a well-known anti-cancer drug which acts by enhancing and stabilizing microtubule formation. Many analogs of Paclitaxel are known, including docetaxel. Docetaxel is also referred to as “Taxotere”. The structures of other paclitaxel analogs are shown in below (and in US Application Publication No. 2006/0135595 the entire contents of which are incorporated herein by reference):

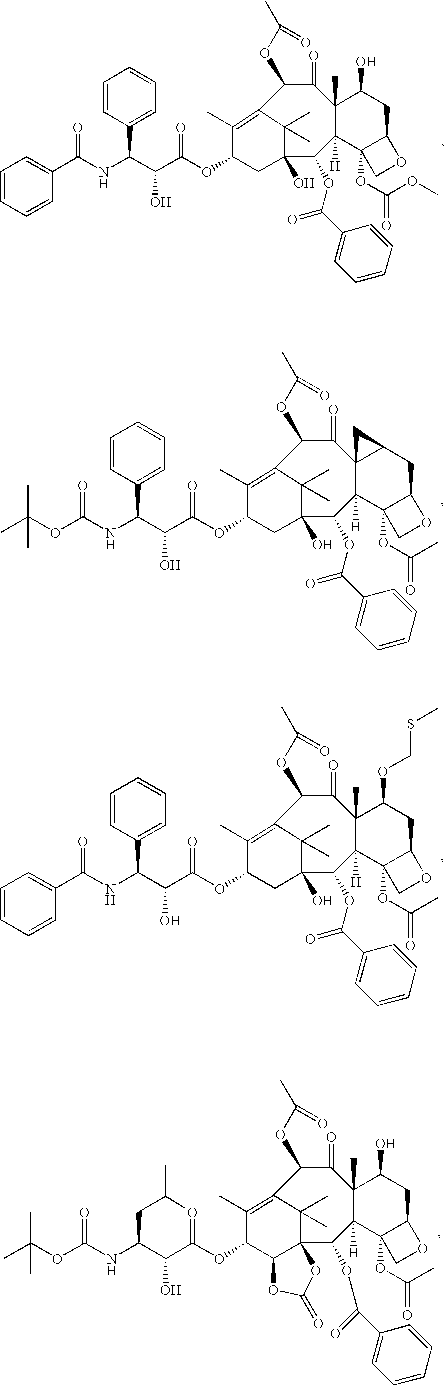

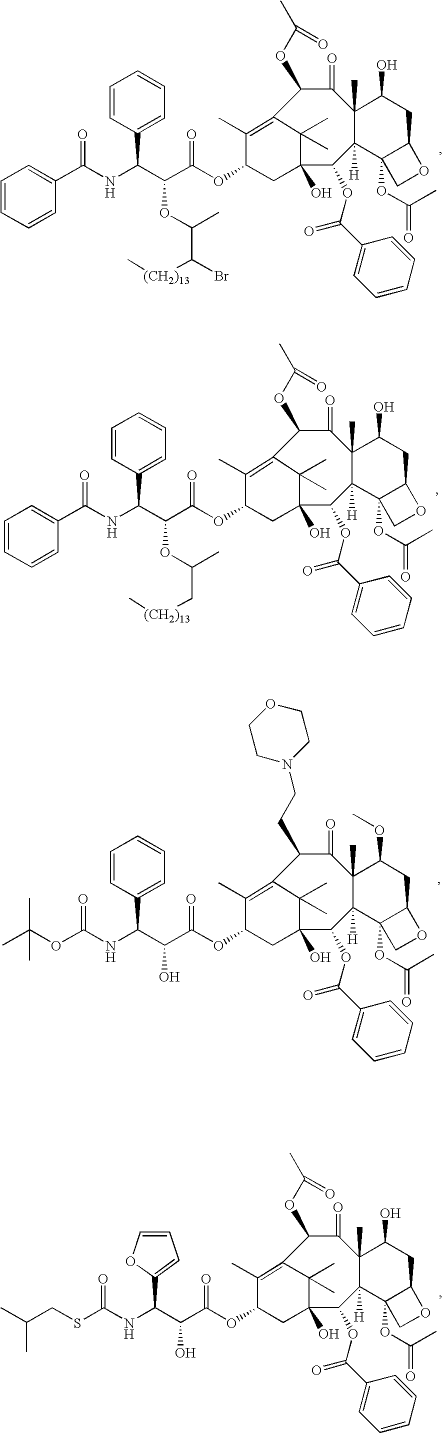

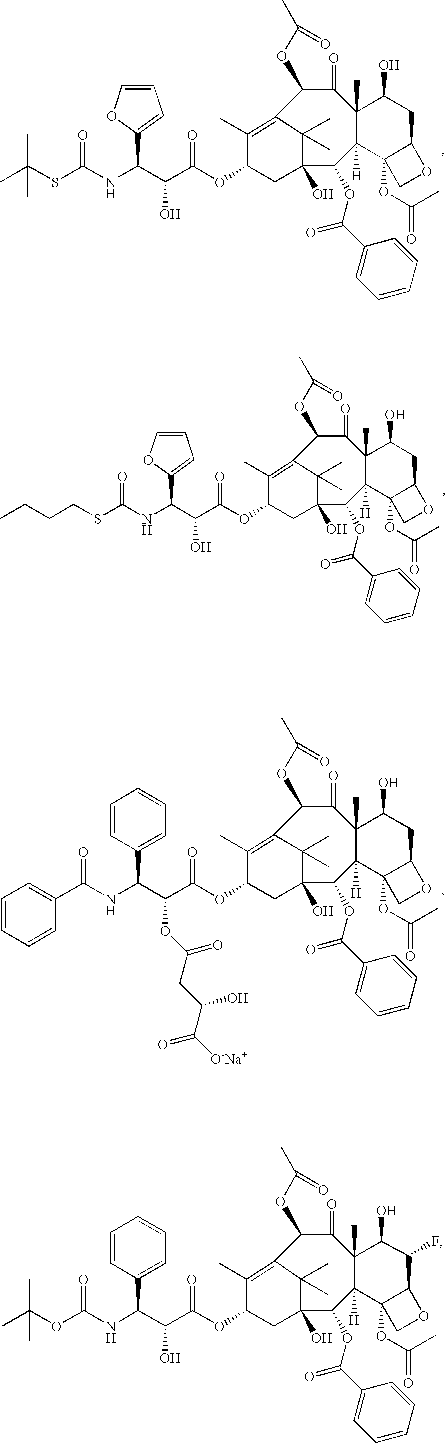

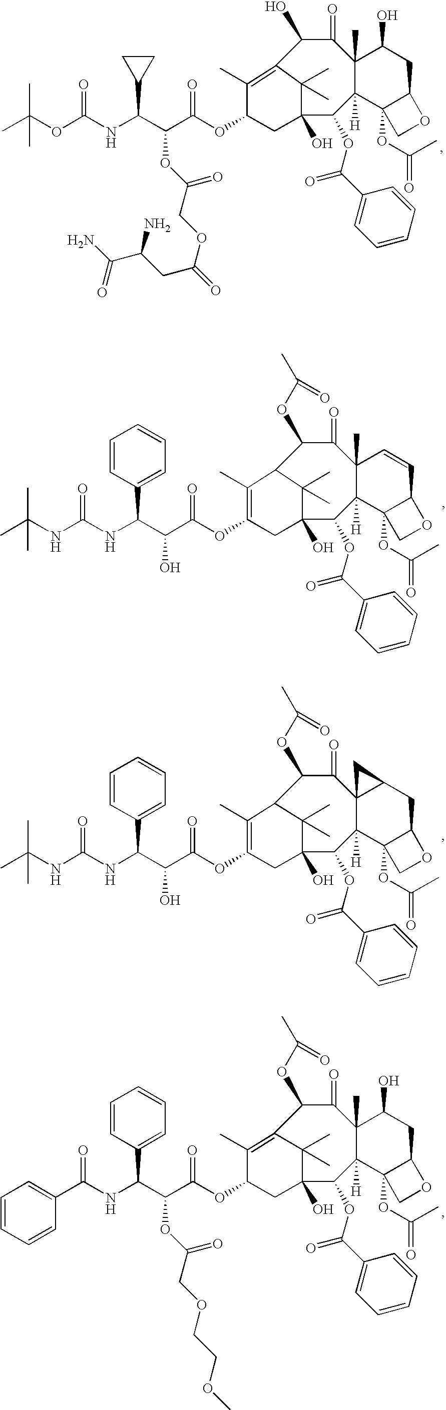

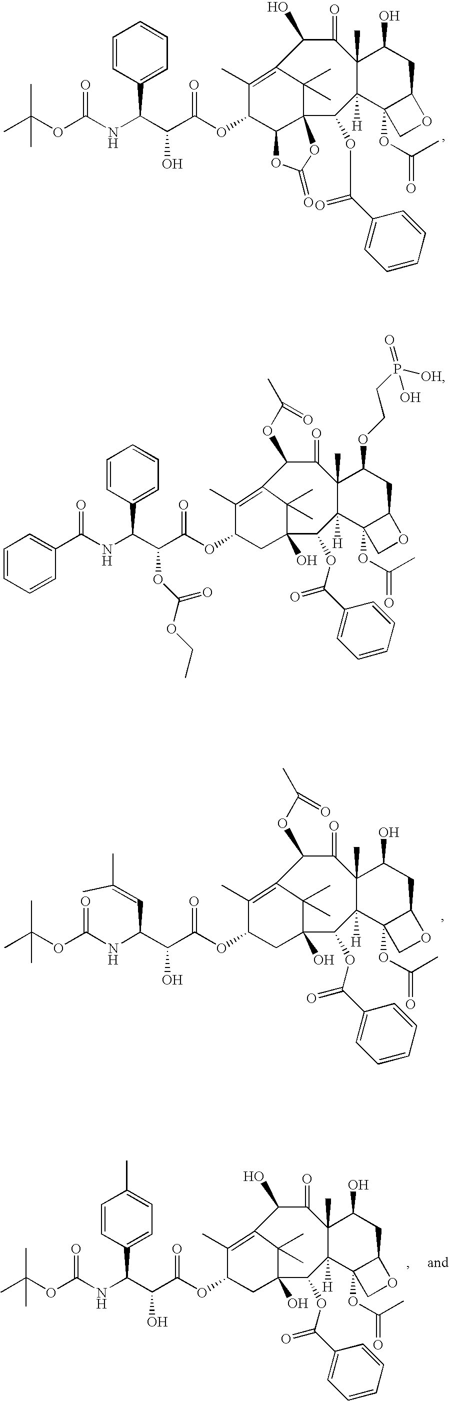

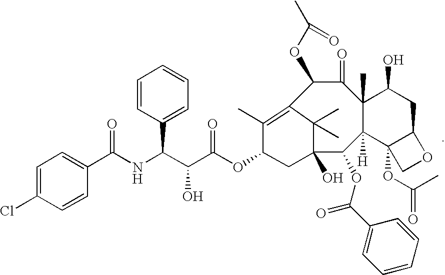

These compounds have the basic taxane skeleton as a common structure feature and have also been shown to have the ability to arrest cells in the G2-M phases due to stabilization of microtubules. Thus, a wide variety of substituents can decorate the taxane skeleton without adversely affecting biological activity. It is also apparent that zero, one or both of the cyclohexane rings of a paclitaxel analog can have a double bond at the indicated positions. For clarity purposes, the basic taxane skeleton is shown below in Structural Formula (X):

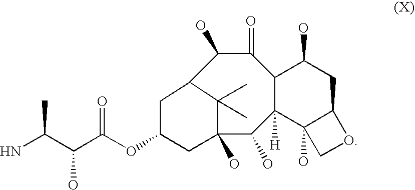

Double bonds have been omitted from the cyclohexane rings in the taxane skeleton represented by Structural Formula (X). The basic taxane skeleton can include zero or one double bond in one or both cyclohexane rings, as indicated in Structural Formulas (XI) and (XII) below. A number of atoms have also been omitted from Structural Formula (X) to indicate sites in which structural variation commonly occurs among paclitaxel analogs. For example, substitution on the taxane skeleton with simply an oxygen atom indicates that hydroxyl, acyl, alkoxy or another oxygen-bearing substituent is commonly found at the site. These and other substitutions on the taxane skeleton can be made without losing the ability to enhance and stabilize microtubule formation. Thus, the term “paclitaxel analog” is defined herein to mean a compound which has the basic paclitaxel skeleton and which promotes microtubule formation. Paclitaxel analogs may be formulated as a nanoparticle colloidal composition to improve the infusion time and to eliminate the need to deliver the drug with Cremophor which causes hypersensitivity reactions in some patients. An example of a paclitaxel analog formulated as a nanoparticle colloidal composition is ABI-007 which is a nanoparticle colloidal composition of protein-stabilized paclitaxel that is reconstituted in saline.

Typically, the paclitaxel analogs used herein are represented by Structural Formula (XI) or (XII):

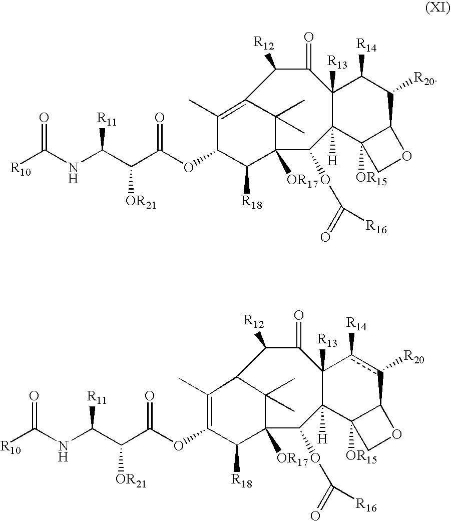

R₁₀ is a lower alkyl group, a substituted lower alkyl group, a phenyl group, a substituted phenyl group, —SR₁₉, —NHR₁₉ or —OR₁₉.

R₁₁ is a lower alkyl group, a substituted lower alkyl group, an aryl group or a substituted aryl group.

R₁₂ is —H, —OH, lower alkyl, substituted lower alkyl, lower alkoxy, substituted lower alkoxy, —O—C(O)-(lower alkyl), —O—C(O)-(substituted lower alkyl), —O—CH₂—O-(lower alkyl)-S—CH₂—O-(lower alkyl).

R₁₃ is —H, —CH₃, or, taken together with R₁₄, —CH₂—.

R₁₄ is —H, —OH, lower alkoxy, —O—C(O)-(lower alkyl), substituted lower alkoxy, —O—C(O)-(substituted lower alkyl), —O—CH₂—O—P(O)(OH)₂, —O—CH₂—O-(lower alkyl), —O—CH₂—S-(lower alkyl) or, taken together with R₂₀, a double bond.

R₁₅—H, lower acyl, lower alkyl, substituted lower alkyl, alkoxymethyl, alkthiomethyl, —OC(O)—O(lower alkyl), —OC(O)—O(substituted lower alkyl), —OC(O)—NH(lower alkyl) or —OC(O)—NH(substituted lower alkyl).

R₁₆ is phenyl or substituted phenyl.

R₁₇ is —H, lower acyl, substituted lower acyl, lower alkyl, substituted, lower alkyl, (lower alkoxy)methyl or (lower alkyl)thiomethyl.

R₁₈—H, —CH₃ or, taken together with R₁₇ and the carbon atoms to which R₁₇ and

R₁₈ are bonded, a five or six membered a non-aromatic heterocyclic ring.

R₁₉ is a lower alkyl group, a substituted lower alkyl group, a phenyl group, a substituted phenyl group.

R₂₀ is —H or a halogen.

R₂₁ is —H, lower alkyl, substituted lower alkyl, lower acyl or substituted lower acyl.

Preferably, the variables in Structural Formulas (XI) and (XII) are defined as follows: R₁₀ is phenyl, tert-butoxy, —S—CH₂—CH—(CH₃)₂, —S—CH(CH₃)₃, —S—(CH₂)₃CH₃, —O—CH(CH₃)₃, —NH—CH(CH₃)₃, —CH═C(CH₃)₂ or para-chlorophenyl; R₁₁ is phenyl, (CH₃)₂CHCH₂—, -2-furanyl, cyclopropyl or para-toluoyl; R₁₂ is —H, —OH, CH₃CO— or —(CH₂)₂—N-morpholino; R₁₃ is methyl, or, R₁₃ and R₁₄, taken together, are —CH₂—;

R₁₄ is —H, —CH₂SCH₃ or —CH₂—O—P(O)(OH)₂; R₁₅ is CH₃CO—;

R₁₆ is phenyl; R₁₇—H, or, R₁₇ and R₁₈, taken together, are —O—CO—O—;

R₁₈ is —H; R₂₀ is —H or —F; and R₂₁ is —H, —C(O)—CHBr—(CH₂)₁₃—CH₃ or —C(O)—(CH₂)₁₄—CH₃; —C(O)—CH₂—CH(OH)—COOH, —C(O)—CH₂—O—C(O)—CH₂CH(NH₂)—CONH₂, —C(O)—CH₂—O—CH₂CH₂OCH₃ or —C(O)—O—C(O)—CH₂CH₃.

A paclitaxel analog can also be bonded to or be pendent from a pharmaceutically acceptable polymer, such as a polyacrylamide. One example of a polymer of this type is shown in U.S. application Ser. No. 11/157,2213. The term “paclitaxel analog”, as it is used herein, includes such polymers.

In some embodiments, paclitaxel anologs have a taxane skeleton represented by Structural Formula IX, wherein Z is O, S, or NR. Paclitaxel anologs that have the taxane skeleton shown in Structural Formula IX can have various substituents attached to the taxane skeleton and can have a double bond in zero, one or both of the cyclohexane rings as shown, for example in FIGS. 3-23.

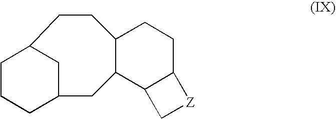

Various paclitaxel analogs and paclitaxel formulations are described in Hennenfent et al. (2006)Annals of Oncology 17:735-749; Gradishar (2006)Expert Opin. Pharmacother. 7(8):1041-53; Attard et al. (2006)Pathol Biol 54(2):72-84; Straubinger et al. (2005)Methods Enzymol. 391:97-117; Ten Tije et al. (2003)Clin Pharmacokinet. 42(7):665-85; and Nuijen et al. (2001) Invest New Drugs. 19(2): 143-53, the entire teachings of which are incorporated herein by reference.

In a particular embodiment of the present invention, the bis(thiohydrazide amides) disclosed herein are administered to a subject suffering from lentigo maligna, superficial spreading malignant melanoma, acral lentiginous malignant melanoma or nodular malignant melanoma in combination with an effective amount of a microtubulin stabilizer (e.g., paclitaxel or docetaxel) and an effective amount of another anti-cancer agent as described herein.

In a particular embodiment, the bis(thiohydrazide amides) are administered in combination with an effective amount of paclitaxel or docetaxel and an effective amount of an anti-cancer agents are selected from the group consisting of dacarbazine (brand name DTIC), temozolomide (brand name Temodar), cisplatin, carmustine (also known as BCNU), fotemustine, vindesine, vincristine sorafenib and bleomycin. In another particular embodiment, the bis(thiohydrazide amides) are administered in combination with an effective amount paclitaxel or docetaxel and an effective amount of an anti-cancer agents are selected from the group carboplatin, tamoxifen and Nolvadex. In another particular embodiment the bis(thiohydrazide amides) are administered in combination with an effective amount of paclitaxel or docetaxel and an effective amount of an anti-cancer agents selected from the group vinablastine, G-CSF and navelbine. In another particular embodiment the bis(thiohydrazide amides) are administered in combination with an effective amount of paclitaxel or docetaxel and an effective amount of an anti-cancer agents selected from the combinations of drugs selected from dacarbazine and G-CSF or carboplatin and sorafenib. In another particular embodiment the bis(thiohydrazide amides) are administered in combination with an effective amount of paclitaxel or docetaxel and an effective amount of an anti-cancer agents selected from the combinations of drugs selected from dacarbazine and Granulocyte colony-stimulating factor (G-CSF), Carboplatin and Sorafenib, dacarbazine, carmustine cisplatin, and tamoxifen, or cisplatin, vinblastine, and dacarbazine.

In a particular embodiment of the present invention, the bis(thiohydrazide amides) disclosed herein are administered to a subject suffering from lentigo maligna, superficial spreading malignant melanoma, acral lentiginous malignant melanoma or nodular malignant melanoma in combination with an effective amount of an anti-cancer agent selected from dacarbazine (brand name DTIC), temozolomide (brand name Temodar), cisplatin, carmustine (also known as BCNU), fotemustine, vindesine, vincristine, bleomycin and combinations thereof. In another particular embodiment the an anti-cancer agent is selected from the group sorafenib, carboplatin, tamoxifen, Nolvadex vinablastine, G-CSF and navelbine.

In another embodiment in the methods of the present invention the bisthiohydrazide amide) is administered in combination with, for example, an effective amount of a combination of dacarbazine, carmustine cisplatin, and tamoxifen, cisplatin, vinblastine, and dacarbazine, or Navelbine and Nolvadex and optionally a microtublin stabilizer.

In a particular embodiment, the bis(thiohydrazide amides) described herein are administered in combination with a biological therapy selected from the group interferons, interleukins, biochemotherapy, vaccine therapy, and antibody-based therapies and optionally a microtublin stabilizer.

In a particular embodiment the bis(thiohydrazide amides) described herein are administered in combination with an anti-angiogenesis therapy selected from the group thalidomide, endostatin and interferon or combination or interferon with other angiogenesis inhibitors, such as thalidomide and endostatin and optionally a microtublin stabilizer.

In certain embodiments of the present invention, the bis(thiohydrazide amides) are administered in combination with a therapy selected from Interleukin2 (IL2; Proleukin), Interferon (IFN alfa-2b, IFN), IFN (interferon) in combination, MDX 010, MDX-1379, Dacarbazide, Genasense, Cisplatin, vinblastine, Carmustine, dacarbazine, or Nolvadex, or selected from the following groups:

### Biologic Response Modifiers:

### Interleukin 2 (IL2; Proleukin)

Interferon (IFN alfa-2b, IFN)

### Biochemotherapy:

IFN (interferon) in combination

### MDX 010+IL-2

### MDX010+MDX-1379

### Dacarbazide+Genasense

### Dacarbazide+Cisplatin+IFN

### Dacarbazide+Cisplatin+IFN+IL-2

Cisplatin+vinblastine+dacarbazine+IL-2+IFN
Carmustine+dacarbazine+cisplatin+Nolvadex+IL-2+IFN

In certain embodiments of the present invention, the bis(thiohydrazide amides) are administered with paclitaxel or docetaxel and a therapy selected from Interleukin2 (IL2; Proleukin), Interferon (IFN alfa-2b, IFN), IFN (interferon) in combination, MDX 010, MDX-1379, Dacarbazide, Genasense, Cisplatin, vinblastine, Carmustine, dacarbazine, or Nolvadex, or selected from the following groups:

### Biologic Response Modifiers:

### Interleukin2 (IL2; Proleukin)

Interferon (IFN alfa-2b, IFN)

### Biochemotherapy:

IFN (interferon) in combination

### MDX 010+IL-2

### MDX010+MDX-1379

### Dacarbazide+Genasense

### Dacarbazide+Cisplatin+IFN

### Dacarbazide+Cisplatin+IFN+IL-2

Cisplatin+vinblastine+dacarbazine+IL-2+IFN
Carmustine+dacarbazine+cisplatin+Nolvadex+IL-2+IFN.

In a preferred embodiment the bis(thiohydrazide amides) described herein are administered in combination with an immunotherapy. Immunotherapy (also called biological response modifier therapy, biologic therapy, biotherapy, immune therapy, or biological therapy) is treatment that uses parts of the immune system to fight disease. Immunotherapy can help the immune system recognize cancer cells, or enhance a response against cancer cells. Immunotherapies include active and passive immunotherapies. Active immunotherapies stimulate the body's own immune system while passive immunotherapies generally use immune system components created outside of the body.

Examples of active immunotherapies include, but are not limited to vaccines including cancer vaccines, tumor cell vaccines (autologous or allogeneic), dendritic cell vaccines, antigen vaccines, anti-idiotype vaccines, DNA vaccines, viral vaccines, or Tumor-Infiltrating Lymphocyte (TIL) Vaccine with Interleukin-2 (IL-2) or Lymphokine-Activated Killer (LAK) Cell Therapy.

Examples of passive immunotherapies include but are not limited to monoclonal antibodies and targeted therapies containing toxins. Monoclonal antibodies include naked antibodies and conjugated antibodies (also called tagged, labeled, or loaded antibodies). Naked monoclonal antibodies do not have a drug or radioactive material attached whereas conjugated monoclonal antibodies are joined to, for example, a chemotherapy drug (chemolabeled), a radioactive particle (radiolabeled), or a toxin (immunotoxin).

In certain embodiments of the present invention passive immunotherapies, such as, naked monoclonal antibody drugs can be used in combination with the bis(thio hydrazide amides) described herein to treat cancer. Examples of these naked monoclonal antibody drugs include, but are not limited to Rituximab (Rituxan), an antibody against the CD20 antigen used to treat, for example, B cell non-Hodgkin lymphoma; Trastuzumab (Herceptin), an antibody against the HER2 protein used to treat, for example, advanced breast cancer; Alemtuzumab (Campath), an antibody against the CD52 antigen used to treat, for example, B cell chronic lymphocytic leukemia (B-CLL); Cetuximab (Erbitux), an antibody against the EGFR protein used, for example, in combination with irinotecan to treat, for example, advanced colorectal cancer and head and neck cancers; and Bevacizumab (Avastin) which is an antiangiogenesis therapy that works against the VEGF protein and is used, for example, in combination with chemotherapy to treat, for example, metastatic colorectal cancer.

Further examples of therapeutic antibodies that can be used include, but are not limited to, HERCEPTIN® (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/Medlmmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CD20-sreptdavidin (+biotin-yttrium 90; NeoRx); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan), and CAT-152 is a human anti-TGF-β₂ antibody (Cambridge Ab Tech).

In certain embodiments of the present invention passive immunotherapies, such as, conjugated monoclonal antibodies can be used in combination with the bis(thio hydrazide amides) described herein to treat cancer. Examples of these conjugated monoclonal antibodies include, but are not limited to Radiolabeled antibody Ibritumomab tiuxetan (Zevalin) which delivers radioactivity directly to cancerous B lymphocytes and is used to treat, for example, B cell non-Hodgkin lymphoma; radiolabeled antibody Tositumomab (Bexxar) which is used to treat, for example, certain types of non-Hodgkin lymphoma; and immunotoxin Gemtuzumab ozogamicin (Mylotarg) which contains calicheamicin and is used to treat, for example, acute myelogenous leukemia (AML). BL22 is a conjugated monoclonal antibody for treating, for example, hairy cell leukemia, immunotoxins for treating, for example, leukemias, lymphomas, and brain tumors, and radiolabeled antibodies such as OncoScint for example, for colorectal and ovarian cancers and ProstaScint for example, for prostate cancers.

In certain embodiments of the present invention targeted therapies containing toxins can be used in combination with the bis(thio hydrazide amides) described herein to treat cancer. Targeted therapies containing toxins are toxins linked to growth factors and do not contain antibodies, for example, denileukin diftitox (Ontak) which can be used to treat, for example, skin lymphoma (cutaneous T cell lymphoma) in combination with the bis(thiohydrazide amides) described herein.

The present invention also includes the use of adjuvant immunotherapies in combination with the bis(thio hydrazide amides) described herein include, such adjuvant immunotherapies include, but are not limited to, cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guérin (BCG); Keyhole limpet hemocyanin (KLH); Incomplete Freund's adjuvant (IFA); QS-21; DETOX; Levamisole; and Dinitrophenyl (DNP), and combinations thereof, such as, for example, combinations of, interleukins, for example, IL-2 with other cytokines, such as IFN-alpha.

In another preferred embodiment the bis(thiohydrazide amides) described herein are administered in combination with an immunotherapy and paclitaxel or docetaxel.

The bis(thio-hydrazide amide) disclosed herein can be prepared by the methods described in U.S. Publication Nos. 20060135595, 2003/0045518 and 2003/0119914, U.S. application Ser. No. 11/432,307, filed 11 May 2006, titled Synthesis Of Bis(Thio-Hydrazide Amide) Salts, U.S. Provisional Patent No. 60/708,977 filed 16 Aug. 2005, titled Bis(Thio-Hydrazide Amide) Formulation and also according to methods described in U.S. Publication No. 2004/0225016 A1, entitled TREATMENT FOR CANCERS. The entire teachings of these applications are incorporated herein by reference.

The present invention is illustrated by the following examples, which are not intended to be limiting in any way.

### EXEMPLIFICATION

Example 1, weekly treatment regimen of compound (1) and paclitaxel combined in Stage IV metastatic melanoma patients in comparison with paclitaxel alone, based on time to progression

A total of 81 people with Stage IV melanoma were tested in a randomized trial with ratios of 2:1, compound (1)+paclitaxel (53 people): paclitaxel alone (28 people). The dosages administered were 213 mg/m² compound (1), 80 mg/m² paclitaxel, and the dosage regimen was 3 weekly doses per each 4 week cycle. Patients were treated until progression of the disease. Patients who progressed on paclitaxel alone were given the option to crossover to compound (1)+paclitaxel and were treated until progression. The tumor assessments were performed at baseline, Cycle 2, and every other Cycle thereafter.

The baseline grades of metastatic diseases of the patients are shown below:

| | compound (1) + | |
|---|---|---|
| | Paclitaxel | Paclitaxel |
| | (n = 53) | (n = 28) |
| M1a - metastasis to distant skin | 7 (13%) | 2 (7%) |
| and subcutaneous tissue | | |
| M1b - metastasis to lungs | 18 (34%) | 5 (18%) |
| M1c - metastasis to other distant | 28 (53%) | 21 (75%) |
| organs, such as liver and brain | | |

Though the majority of the patients in the paclitaxel alone treatment group were M1c, an analysis of the effect of M grade did not show a statistically significant effect on the patient's likelihood of progressing more quickly (p-value=0.5368). The actual treatment the patient received did have a statistically significant effect on the patient's likelihood of progressing more quickly (p-value=0.0281).

The probability-value for the continuum of potential outcomes was divided into four scenarios from best to worst:

i) Inverted or Equal results;

ii) 4783 better p>0.2;

iii) Favorable 0.05≦p≦0.2 to; and

iv) Favorable p<0.05.

Table I shows the Kaplier Meyer estimates of the Time to Progression of the disease (Efficacy Sample):

TABLE 1

| | compound (1) + | | | | |
|---|---|---|---|---|---|
| Time to Progression | Paclitaxel | | Paclitaxel | | |
| (days) | (n = 50) | | (n = 27) | | p-value* |
| 25^{th} percentile (95% | 54.0 | (49.0, 95.0) | 49.0 | (29.0, 52.0) | 0.017 |
| confidence interval (CI)) | | | | | |
| Median (95% CI) | 134.0 | (86.0, 217.0 | 56.0 | (49.0, 105.0) | |
| 75^{th} percentile (95% CI) | 273.0 | (168.0, 331.0) | 106.0 | (61.0, 218.0) | |

The p-value is from a log-rank test

Based on the four scenarios above the study results are in line with the best of the four possible scenarios.

Table 2 shows the best overall response per Response Evaluation Criteria In Solid Tumors (RECIST) (Efficacy Sample)

TABLE 2

| | compound (1) + | | | | |
|---|---|---|---|---|---|
| | Paclitaxel | | Paclitaxel | | |
| | (n = 50) | | (n = 27) | | p-value* |
| Best Overall Response | | | | | |
| Complete Response (CR) | 1 | (2.0%) | 0 | | |
| Partial Response (PR) | 7 | (14.0%) | 1 | (3.7%) | |
| Stable Disease (SD) | 25 | (50.0%) | 10 | (37%) | |
| Progressive Disease (PD) | 17 | (34.0%) | 16 | (59.3%) | |
| Two-Sided Fisher's Exact | | | | | |
| Test | | | | | |
| CR + PR (95% CI) | 16.0% | (7.2%, 29.1%) | 3.7% | (0.1%, 19.0%) | 0.149 |

As can be seen from Table 2 compounds of the present invention in combination with paclitaxel show a significant improvement over paclitaxel alone. Specifically compounds of the present invention in combination with paclitaxel showed one patent with a complete response and over 50% of the patients had stable disease compared with Paclitaxel alone which only showed 37% of the patients with stable disease.

Tables 3 and 4 show the relative treatment results of compound (1) in combination with Paclitaxel compared with Paclitaxel alone and other currently used treatments for melanoma. As can be seen from Tables 3 and 4 the number of days to progression of the disease is greatly enhanced for compound (1) in combination with Paclitaxel compared with Paclitaxel alone. In addition the time to progression benefit is much better than any single-agent therapy and much better than all but one combination therapy currently used.

The combination therapy, cisplatin vinblastine dacarbazine IL-2 and IFN, which had a longer time to progression than compound (1) in combination with Paclitaxel, however, has severe side effects and requires patients to be hospitalized for administration of the combination. Conversely, compound (1) in combination with Paclitaxel only showed a mild increase in the side effects over Paclitaxel alone. None of the side effect were sever enough to cause any patients to discontinue treatment with compound (1) in combination with Paclitaxel during the trial.

TABLE 3

| | CR | PR | OR | TTP | Survival |
|---|---|---|---|---|---|
| Agent/Regimen | (%) | (%) | (%) | (days) | (months) |
| Natural disease | | | | | 6-9 |
| progression | | | | | |
| “Any Treatment” | | | 5-10 | | |

Single Agent Chemotherapy

| | | | | | |
|---|---|---|---|---|---|
| DTIC (dacarbazine) | rare | | 10-20 | | no |
| | ≦3 | | | | improvement |
| Temozolomide | 2.6 | 9.6% | 13.5 | 58 | 7.7 |
| (Temodar) | | | | | |
| Paclitaxel (Taxol) | | | 12, 17.8 | | |
| Paclitaxel | 0 | 3.7 | 3.7 | 57 | N.D. |
| Fotemustine | | | 15.2 | 55 | 7.3 |
| Sorafenib | | 2.6 | | | |

Anti-Estrogen Therapy

| | | | |
|---|---|---|---|
| Tamoxifen | 1 | 3.9 | 4.9 |

TABLE 4

| | CR | PR | OR | TTP | Survival |
|---|---|---|---|---|---|
| Agent/Regimen | (%) | (%) | (%) | (days) | (months) |
| Natural disease progression | | | | | 6-9 |
| “Any Treatment” | | | 5-10 | | |

Biological Response Modifiers

| | | | | |
|---|---|---|---|---|
| Interleukin-2 | 6 | 10 | 14.3, 16 | 8.7, ≦12 |
| (IL-2; Proleukin ®⁾ | | | | |
| Interferon | 3-5 | | 15 | |
| (IF N alfa-2b, IF N) | | | | |

Biochemotherapy

| | | | | | |
|---|---|---|---|---|---|
| IFN in combination | | | 24 | | |
| MDX-010 + IL-2 | 5.6 | 16.7 | 22.2 | | |
| MDX-010 + MDX-1379 | 3.6 | 8.9 | 12.5 | | |
| Dacarbazide + Genasense | | | 11.7 | 78 | 9.1 |
| Dacarbazide + Cisplatin + | | | | 92 | 9 |
| IFN | | | | | |
| Dacarbazide + Cisplatin + | | | | 119 | 9 |
| IFN + IL-2 | | | | | |
| Paclitaxel + compound (1) | 2.0 | 14.0 | 16 | 134 | N.D. |
| Cisplatin + vinblastine + | 6.6 | | | 149 | 11.9 |
| dacarbazine + IL-2 + IFN | | | | | |
| Carmustine + dacarbazine + | 13 | 30 | 43 | | |
| cisplatin + Nolvadex + | | | | | |
| IL-2 + IFN | | | | | |

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating a subject with lentigo maligna, comprising administering to the subject an effective amount of a compound represented by the following Structural Formula: 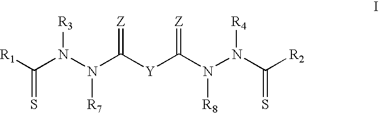
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group; and
Z is O or S.

2.-20. (canceled)

21. The method of claim 1, wherein the compound is a disodium or a dipotassium salt.

22. The method of claim 1, wherein the subject is suffering from Stage IV lentigo maligna.

23. The method of claim 1, wherein the compound is administered in combination with an effective amount of a microtubulin stabilizer selected from the group consisting of paclitaxel, paclitaxel analogues, Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof.

24.-28. (canceled)

29. The method of claim 1 wherein the compound is co-administered with an effective amount of paclitaxel or docetaxel.

30. The method of claim 29, wherein the compound is further co-administered with an effective amount of an anti-cancer-agent selected from the group consisting of dacarbazine, temozolomide, cisplatin, carmustine, fotemustine, vindesine, vincristine, vinablastine, G-CSF, navelbine, tamoxifen, carboplatin, nolvadex, sorafenib, bleomycin and combinations thereof.

31. (canceled)

32. The method of claim 1, wherein the compound is co-administered with an effective amount of an anti-cancer-agent selected from the group consisting of dacarbazine, temozolomide, cisplatin, carmustine, fotemustine, vindesine, vincristine, vinablastine, G-CSF, navelbine, tamoxifen, carboplatin, nolvadex, sorafenib or bleomycin.

33. (canceled)

34. A method of treating a subject with lentigo maligna, comprising administering to the subject an effective amount of a compound represented by a Structural Formula selected from: 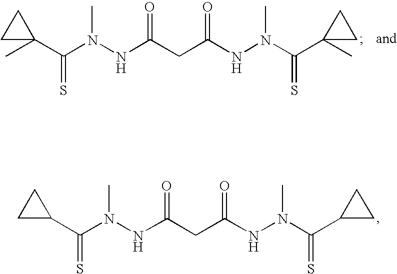
or a pharmaceutically acceptable salt thereof.

35.-42. (canceled)

43. A method of treating a subject with lentigo maligna, comprising administering to the subject an effective amount of a compound represented by the following Structural Formula: 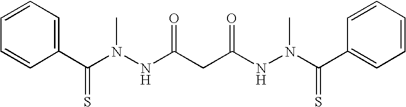
or a pharmaceutically acceptable salt thereof; in combination with an effective amount of paclitaxel or docetaxel.

44.-45. (canceled)

46. A method of preventing or delaying the recurrence of melanoma in a subject who has been treated for lentigo maligna, comprising administering to the subject an effective amount of a compound represented by the following
Structural Formula: 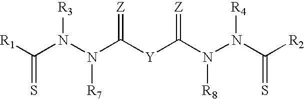
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group; and
Z is O or S.

47. A method of treating a subject with superficial spreading malignant melanoma, comprising administering to the subject an effective amount of a compound represented by the following Structural Formula: 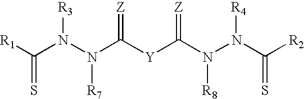
or a pharmaceutically acceptable salt or solvate thereof, wherein:
Y is a covalent bond or an optionally-substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group; and
Z is O or S.

48.-66. (canceled)

67. The method of claim 47, wherein the compound is a disodium or a dipotassium salt.

68. The method of claim 47, wherein the subject is suffering from Stage IV superficial spreading malignant melanoma.

69. The method of claim 47, wherein the compound is administered in combination with an effective amount of a microtubulin stabilizer selected from the group consisting of paclitaxel, paclitaxel analogues, Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof.

70.-74. (canceled)

75. The method of claim 47 wherein the compound is co-administered with an effective amount of paclitaxel or docetaxel.

76. The method of claim 75, wherein the compound is further co-administered with an effective amount of an anti-cancer-agent selected from the group consisting of dacarbazine, temozolomide, cisplatin, carmustine, fotemustine, vindesine, vincristine, vinablastine, G-CSF, navelbine, tamoxifen, carboplatin, nolvadex, sorafenib, bleomycin and combinations thereof.

77. (canceled)

78. The method of claim 47, wherein the compound is co-administered with an effective amount of an anti-cancer-agent selected from the group consisting of dacarbazine, temozolomide, cisplatin, carmustine, fotemustine, vindesine, vincristine, vinablastine, G-CSF, navelbine, tamoxifen, carboplatin, nolvadex, sorafenib or bleomycin.

79. (canceled)

80. A method of treating a subject with superficial spreading malignant melanoma, comprising administering to the subject an effective amount of a compound represented by a Structural Formula selected from: 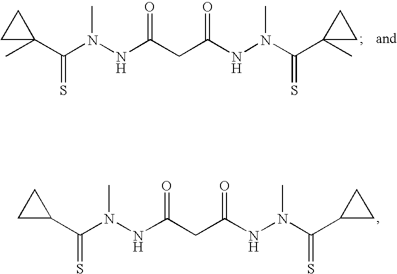
or a pharmaceutically acceptable salt thereof.

81.-88. (canceled)

89. A method of treating a subject with superficial spreading malignant melanoma, comprising administering to the subject an effective amount of a compound represented by the following Structural Formula: 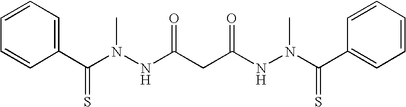
or a pharmaceutically acceptable salt thereof; in combination with an effective amount of paclitaxel or docetaxel.

90.-91. (canceled)

92. A method of preventing or delaying the recurrence of melanoma in a subject who has been treated for superficial spreading malignant melanoma, comprising administering to the subject an effective amount of a compound represented by the following Structural Formula: 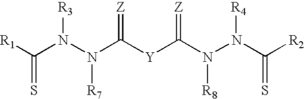
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group; and
Z is O or S.

93. A method of treating a subject with acral lentiginous malignant melanoma comprising administering to the subject an effective amount of a compound represented by the following Structural Formula: 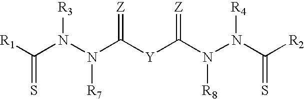
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group; and
Z is O or S.

94.-112. (canceled)

113. The method of claim 93, wherein the compound is a disodium or a dipotassium salt.

114. The method of claim 93, wherein the subject is suffering from Stage IV acral lentiginous malignant melanoma.

115. The method of claim 93, wherein the compound is administered in combination with an effective amount of a microtubulin stabilizer selected from the group consisting of paclitaxel, paclitaxel analogues, Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin;
Taccalonolide; Eleutherobin; Sarcodictyin; Laulimalide; Dictyostatin-1;
Jatrophane esters; and analogs and derivatives thereof.

116.-120. (canceled)

121. The method of claim 93 wherein the compound is co-administered with an effective amount of paclitaxel or docetaxel.

122. The method of claim 121, wherein the compound is further co-administered with an effective amount of an anti-cancer-agent selected from the group consisting of dacarbazine, temozolomide, cisplatin, carmustine, fotemustine, vindesine, vincristine, vinablastine, G-CSF, navelbine, tamoxifen, carboplatin, nolvadex, sorafenib, bleomycin and combinations thereof.

123. (canceled)

124. The method of claim 93, wherein the compound is co-administered with an effective amount of an anti-cancer-agent selected from the group consisting of dacarbazine, temozolomide, cisplatin, carmustine, fotemustine, vindesine, vincristine, vinablastine, G-CSF, navelbine, tamoxifen, carboplatin, nolvadex, sorafenib or bleomycin.

125. (canceled)

126. A method of treating a subject with acral lentiginous malignant melanoma comprising administering to the subject an effective amount of a compound represented by a Structural Formula selected from: 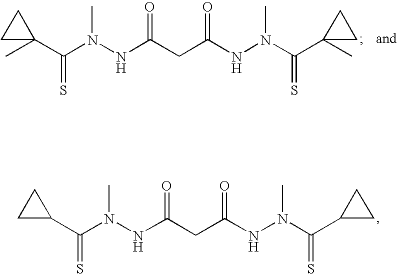
or a pharmaceutically acceptable salt thereof.

127-134. (canceled)

135. A method of treating a subject with acral lentiginous malignant comprising administering to the subject an effective amount of a compound represented by the following Structural Formula: 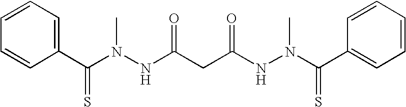
or a pharmaceutically acceptable salt thereof; in combination with an effective amount of paclitaxel or docetaxel.

136-137. (canceled)

138. A method of preventing or delaying the recurrence of melanoma in a subject who has been treated for acral lentiginous malignant melanoma comprising administering to the subject an effective amount of a compound represented by the following Structural Formula: 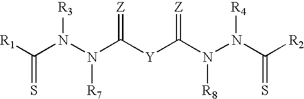
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group; and
Z is O or S.

139. A method of treating a subject with nodular malignant melanoma, comprising administering to the subject an effective amount of a compound represented by the following Structural Formula: 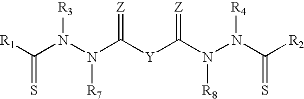
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group; and
Z is O or S.

140.-158. (canceled)

159. The method of claim 139, wherein the compound is a disodium or a dipotassium salt.

160. The method of claim 139, wherein the subject is suffering from Stage IV nodular malignant melanoma.

161. The method of claim 139, wherein the compound is administered in combination with an effective amount of a microtubulin stabilizer selected from the group consisting of paclitaxel, paclitaxel analogues, Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof.

162.-166. (canceled)

167. The method of claim 139, wherein the compound is co-administered with an effective amount of paclitaxel or docetaxel.

168. The method of claim 167, wherein the compound is further co-administered with an effective amount of an anti-cancer-agent selected from the group consisting of dacarbazine, temozolomide, cisplatin, carmustine, fotemustine, vindesine, vincristine, vinablastine, G-CSF, navelbine, tamoxifen, carboplatin, nolvadex, sorafenib, bleomycin and combinations thereof.

169. (canceled)

170. The method of claim 139, wherein the compound is co-administered with an effective amount of an anti-cancer-agent selected from the group consisting of dacarbazine, temozolomide, cisplatin, carmustine, fotemustine, vindesine, vincristine, vinablastine, G-CSF, navelbine, tamoxifen, carboplatin, nolvadex, sorafenib or bleomycin.

171. (canceled)

172. A method of treating a subject with nodular malignant melanoma, comprising administering to the subject an effective amount of a compound represented by a Structural Formula selected from: 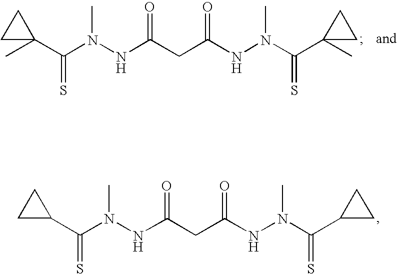
or a pharmaceutically acceptable salt thereof.

173.-180. (canceled)

181. A method of treating a subject with nodular malignant melanoma, comprising administering to the subject an effective amount of a compound represented by the following Structural Formula: 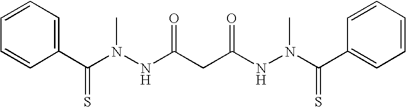
or a pharmaceutically acceptable salt thereof; in combination with an effective amount of paclitaxel or docetaxel.

182.-183. (canceled)

184. A method of preventing or delaying the recurrence of melanoma in a subject who has been treated for nodular malignant melanoma, comprising administering to the subject an effective amount of a compound represented by the following Structural Formula: 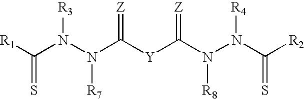
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group; and
Z is O or S.

185. The method of claim 1, further comprising administering an immunotherapy.

186-191. (canceled)

192. The method of claim 47, further comprising administering an immunotherapy.

193. The method of claim 93, further comprising administering an immunotherapy.

194. The method of claim 139, further comprising administering an immunotherapy.
